(19) United States
(12) Patent Application Publication
(10) Pub. No.: US 2006/0257851 A1
(43) Pub. Date: Nov. 16, 2006

(54) Bioinformatically detectable group of novel viral regulatory genes and uses thereof

(76) Inventor: Itzhak Bentwich, Rehovot (IL)

Correspondence Address: HOWREY LLP, C/O IP DOCKETING DEPARTMENT, 2941 FAIRVIEW PARK DR, SUITE 200, FALLS CHURCH, VA, 22042-2924, US

(21) Appl. No.: 10/536,560

(22) PCT Filed: Nov. 26, 2003
(86) PCT No.: PCT/IL03/00998

§ 371 Date: Dec. 20, 2005

Related U.S. Application Data
(63) Continuation-in-part of application No. 10/308,778, filed on Dec. 3, 2002.
Application No. 10/308,778 is a division of application No. 09/522,872, filed on Mar. 10, 2000, now Pat. No. 6,599,723.
Application No. PCT/IL03/00998 is a continuation-in-part of application No. 10/310,188, filed on Dec. 5, 2002, now abandoned.
Application No. 10/310,188 is a continuation-in-part of application No. 10/303,778, filed on Nov. 26, 2002, now abandoned.
Application No. 10/310,188 is a continuation-in-part of application No. 10/604,945, filed on Aug. 27, 2003.
Application No. 10/604,945 is a continuation of application No. 10/303,778, filed on Nov. 26, 2002, now abandoned.
Application No. PCT/IL03/00998 is a continuation-in-part of application No. 10/604,942, filed on Aug. 28, 2003.
Application No. 10/604,942 is a continuation of application No. 10/310,188, filed on Dec. 5, 2002, now abandoned.
Application No. PCT/IL03/00998 is a continuation-in-part of application No. 10/604,943, filed on Aug. 28, 2003.
Application No. PCT/IL03/00998 is a continuation-in-part of application No. 10/604,944, filed on Aug. 28, 2003.
Application No. PCT/IL03/00998 is a continuation-in-part of application No. 10/604,984, filed on Aug. 29, 2003.
Application No. PCT/IL03/00998 is a continuation-in-part of application No. 10/605,838, filed on Oct. 30, 2003.
Application No. 10/605,838 is a continuation of application No. 10/604,944, filed on Aug. 28, 2003.
Application No. 10/605,838 is a continuation-in-part of application No. 10/303,778, filed on Nov. 26, 2002, now abandoned.
Application No. 10/605,838 is a continuation-in-part of application No. 10/310,188, filed on Dec. 5, 2002, now abandoned.
Application No. 10/605,838 is a continuation-in-part of application No. 10/604,945, filed on Aug. 27, 2003.
Application No. 10/605,838 is a continuation-in-part of application No. 10/604,942, filed on Aug. 28, 2003.
Application No. 10/605,838 is a continuation-in-part of application No. 10/604,943, filed on Aug. 28, 2003.
Application No. 10/605,838 is a continuation-in-part of application No. 10/604,984, filed on Aug. 29, 2003.
Application No. PCT/IL03/00998 is a continuation-in-part of application No. 10/605,840, filed on Oct. 30, 2003.
(60) Provisional application No. 60/123,833, filed on Mar. 11, 1999.
(60) Provisional application No. 60/441,241, filed on Jan. 17, 2003.
(60) Provisional application No. 60/411,230, filed on Sep. 17, 2002.
(60) Provisional application No. 60/457,788, filed on Mar. 27, 2003.
(60) Provisional application No. 60/441,241, filed on Jan. 17, 2003.
(60) Provisional application No. 60/411,230, filed on Sep. 17, 2002.
(60) Provisional application No. 60/457,788, filed on Mar. 27, 2003.
(60) Provisional application No. 60/411,230, filed on Sep. 17, 2002.
(60) Provisional application No. 60/441,241, filed on Jan. 17, 2003.
(60) Provisional application No. 60/457,788, filed on Mar. 27, 2003.

(51) Int. Cl. C12Q 1/70; C07H 21/04

(52) U.S. Cl. 435/5; 536/23.72

(57) ABSTRACT

The present invention relates to a first group of novel genes, here identified as genomic address messenger or VGAM genes, and a second group of novel operon-like genes, here identified as viral genomic record or VGR genes. VGAM genes selectively inhibit translation of known ‘target’ genes, many of which are known to be involved in various diseases. Nucleic acid molecules are provided respectively encoding 1560 VGAM genes, and 205 VGR genes, as are vectors and probes both comprising the nucleic acid molecules, and methods and systems for detecting VGAM and VGR genes and specific functions and utilities thereof, for detecting expression of VGAM and VGR genes, and for selectively enhancing and selectively inhibiting translation of the respective target genes thereof.

### BACKGROUND OF THE INVENTION

1. Field of the Invention

The present invention relates to a group of bioinformatically detectable novel viral genes, here identified as Viral Genomic Address Messenger or VGAM genes, which are believed to be related to the micro RNA (miRNA) group of genes.

2. Description of Prior Art

MIR genes are regulatory genes encoding MicroRNA's (miRNA), short ˜22 nt non-coding RNA's, found in a wide range of species, believed to function as specific gene translation repressors, sometimes involved in cell-differentiation. Some 110 human MIR genes have been detected by laboratory means. Over the past 6 months, the need for computerized detection of MIR genes has been recognized, and several informatic detection engines have been reported (Lim, 2003; Grad, 2003; Lai, 2003). Collectively these informatic detection engines found 38 more human MIR genes which were later confirmed in zebrafish, 14 human MIR genes which were confirmed in human, and 55 postulated human MIR genes which could not be confirmed by laboratory (Lim, 2003). Extensive efforts to identify novel MIR genes using conventional biological detection techniques such as massive cloning and sequencing efforts, and several bioinformatic detection attempts, have led leading researchers in the field to the conclusion that the total number of human MIR genes is between 200 to 255 (Lau, 2003; Lim 2003 Science; Lim, 2003 Genes Dev). Recent studies postulate that the number of MIR genes may be higher (Grad, 2003; Krichevsky, 2003).

The ability to detect novel MIR genes is limited by the methodologies used to detect such genes. All MIR genes identified so far either present a visibly discernable whole body phenotype, as do Lin-4 and Let-7 (Wightman, 1993; Reinhart, 2000), or produce sufficient quantities of RNA so as to be detected by the standard molecular biological techniques.

Initial studies reporting MIR genes (Bartel, 2001; Tuschl, 2001) discovered 93 MIR genes in several species, by sequencing a limited number of clones (300 by Bartel and 100 by Tuschl) of small segments (i.e. size fractionated) RNA. MiRNA encoded by MIR genes detected in these studies therefore, represent the more prevalent among the miRNA gene family, and can not be much rarer than 1% of all small ˜20 nt-long RNA segments.

Current methodology has therefore been unable to detect micro RNA genes (MIR genes) which either do not present a visually discernable whole body phenotype, or are rare (e.g. rarer than 0.1% of all size fractionated ˜20 nt-long RNA segments expressed in the tissues examined), and therefore do not produce significant enough quantities of RNA so as to be detected by standard biological techniques. To date, miRNA have not been detected in viruses.

### BRIEF DESCRIPTION OF SEQUENCE LISTING, LARGE TABLES AND COMPUTER PROGRAM LISTING

Sequence listing, large tables related to sequence listing, and computer program listing are filed under section 801 (a)(i) on an electronic medium in computer readable form, attached to the present invention, and are hereby incorporated by reference. Said sequence listing, large tables related to sequence listing, and computer program listing are submitted on a CD-ROM (Operating system: MS-Windows), entitled SEQUENCE LISTING AND LARGE TABLES, containing files the names and sizes of which are as follows:

Sequence listing comprising 424,595 genomic sequences, is filed under section 801 (a)(i) on an electronic medium in computer readable form, attached to the present invention, and is hereby incorporated by reference. Said sequence listing is contained in a self extracting compressed file named SEQ_LIST.EXE (8,389 KB). Compressed file contains 1 file named SEQ_LIST.TXT (63,778 KB).

Large tables relating to genomic sequences are stored in 7 self extracting files, each comprising a respective one of the following table files: TABLE1.TXT (229 KB); TABLE2.TXT (878 KB); TABLE3.TXT (147 KB); TABLE4.TXT (353,556 KB); TABLE5.TXT (868,334 KB); TABLE6.TXT (208,163 KB); and TABLE7.TXT (52 KB).

It is appreciated that the nucleotide ‘U’ is represented as ‘T’ in the sequences incorporated in the enclosed large tables.

Computer program listing of a computer program constructed and operative in accordance with a preferred embodiment of the present invention is enclosed on an electronic medium in computer readable form, and is hereby incorporated by reference. The computer program listing is contained in a self extracting compressed file named COMPUTER PROGRAM LISTING.EXE (100 KB). Compressed file contains 7 files, the name and sizes of which are as follows: AUXILARY_FILES.TXT (117 KB); BINDING_SITE_SCORING.TXT (17 KB); EDIT_DISTANCE.TXT (104 KB); FIRST-K.TXT (48K); HAIRPIN_PREDICTIO.TXT (47 KB); TWO_PHASED_PREDICTOR.TXT (74 KB); and TWO_PHASED_SIDE_SELECTOR.TXT (4 KB).

### SUMMARY OF THE INVENTION

The present invention relates to a novel group of bioinformatically detectable, viral regulatory RNA genes, which repress expression of host target host genes, by means of complementary hybridization to binding sites in untranslated regions of these host target host genes. It is believed that this novel group of viral genes represent a pervasive viral mechanism of attacking hosts, and that therefore knowledge of this novel group of viral genes may be useful in preventing and treating viral diseases.

In various preferred embodiments, the present invention seeks to provide improved method and system for detection and prevention of viral disease, which is mediated by this group of novel viral genes.

Accordingly, the invention provides several substantially pure nucleic acids (e.g., genomic nucleic acid, cDNA or synthetic nucleic acid) each encoding a novel viral gene of the VGAM group of gene, vectors comprising the nucleic acids, probes comprising the nucleic acids, a method and system for selectively modulating translation of known “target” genes utilizing the vectors, an1d a method and system for detecting expression of known “target” genes utilizing the probe.

By “substantially pure nucleic acid” is meant nucleic acid that is free of the genes which, in the naturally-occurring genome of the organism from which the nucleic acid of the invention is derived, flank the genes discovered and isolated by the present invention. The term therefore includes, for example, a recombinant nucleic acid which is incorporated into a vector, into an autonomously replicating plasmid or virus, or into the genomic nucleic acid of a prokaryote or eukaryote at a site other than its natural site; or which exists as a separate molecule (e.g., a cDNA or a genomic or cDNA fragment produced by PCR or restriction endonuclease digestion) independent of other sequences. It also includes a recombinant nucleic acid which is part of a hybrid gene encoding additional polypeptide sequence.

“Inhibiting translation” is defined as the ability to prevent synthesis of a specific protein encoded by a respective gene, by means of inhibiting the translation of the mRNA of this gene. “Translation inhibiter site” is defined as the minimal nucleic acid sequence sufficient to inhibit translation.

There is thus provided in accordance with a preferred embodiment of the present invention a bioinformatically detectable novel viral gene encoding substantially pure nucleic acid wherein: RNA encoded by the bioinformatically detectable novel viral gene is about 18 to about 24 nucleotides in length, and originates from an RNA precursor, which RNA precursor is about 50 to about 120 nucleotides in length, a nucleotide sequence of a first half of the RNA precursor is a partial inversed-reversed sequence of a nucleotide sequence of a second half thereof, a nucleotide sequence of the RNA encoded by the novel viral gene is a partial inversed-reversed sequence of a nucleotide sequence of a binding site associated with at least one host target gene, and a function of the novel viral gene is bioinformatically deducible.

There is further provided in accordance with another preferred embodiment of the present invention a method for anti-viral treatment comprising neutralizing said RNA.

Further in accordance with a preferred embodiment of the present invention the neutralizing comprises: synthesizing a complementary nucleic acid molecule, a nucleic sequence of which complementary nucleic acid molecule is a partial inversed-reversed sequence of said RNA, and transfecting host cells with the complementary nucleic acid molecule, thereby complementarily binding said RNA.

Further in accordance with a preferred embodiment of the present invention the neutralizing comprises immunologically neutralizing.

There is still further provided in accordance with another preferred embodiment of the present invention a bioinformatically detectable novel viral gene encoding substantially pure nucleic acid wherein: RNA encoded by the bioinformatically detectable novel viral gene includes a plurality of RNA sections, each of the RNA sections being about 50 to about 120 nucleotides in length, and including an RNA segment, which RNA segment is about 18 to about 24 nucleotides in length, a nucleotide sequence of a first half of each of the RNA sections encoded by the novel viral gene is a partial inversed-reversed sequence of nucleotide sequence of a second half thereof, a nucleotide sequence of each of the RNA segments encoded by the novel viral gene is a partial inversed-reversed sequence of the nucleotide sequence of a binding site associated with at least one target host gene, and a function of the novel viral gene is bioinformatically deducible from the following data elements: the nucleotide sequence of the RNA encoded by the novel viral gene, a nucleotide sequence of the at least one target host gene, and function of the at least one target host gene.

Further in accordance with a preferred embodiment of the present invention the function of the novel viral gene is bioinformatically deducible from the following data elements: the nucleotide sequence of the RNA encoded by the bioinformatically detectable novel viral gene, a nucleotide sequence of the at least one target host gene, and a function of the at least one target host gene.

Still further in accordance with a preferred embodiment of the present invention the RNA encoded by the novel viral gene complementarily binds the binding site associated with the at least one target host gene, thereby modulating expression of the at least one target host gene.

Additionally in accordance with a preferred embodiment of the present invention the binding site associated with at least one target host gene is located in an untranslated region of RNA encoded by the at least one target host gene.

Moreover in accordance with a preferred embodiment of the present invention the function of the novel viral gene is selective inhibition of translation of the at least one target host gene, which selective inhibition includes complementary hybridization of the RNA encoded by the novel viral gene to the binding site.

Further in accordance with a preferred embodiment of the present invention the invention includes a vector including the DNA.

Still further in accordance with a preferred embodiment of the present invention the invention includes a method of selectively inhibiting translation of at least one gene, including introducing the vector.

Moreover in accordance with a preferred embodiment of the present invention the introducing includes utilizing RNAi pathway.

Additionally in accordance with a preferred embodiment of the present invention the invention includes a gene expression inhibition system including: the vector, and a vector inserter, functional to insert the vector into a cell, thereby selectively inhibiting translation of at least one gene.

Further in accordance with a preferred embodiment of the present invention the invention includes a probe including the DNA.

Still further in accordance with a preferred embodiment of the present invention the invention includes a method of selectively detecting expression of at least one gene, including using the probe.

Additionally in accordance with a preferred embodiment of the present invention the invention includes a gene expression detection system including: the probe, and a gene expression detector functional to selectively detect expression of at least one gene.

Further in accordance with a preferred embodiment of the present invention the invention includes an anti-viral substance capable of neutralizing the RNA.

Still further in accordance with a preferred embodiment of the present invention the neutralizing includes complementarily binding the RNA.

Additionally in accordance with a preferred embodiment of the present invention the neutralizing includes immunologically neutralizing.

Moreover in accordance with a preferred embodiment of the present invention the invention includes a method for anti-viral treatment including neutralizing the RNA.

Further in accordance with a preferred embodiment of the present invention the neutralizing includes: synthesizing a complementary nucleic acid molecule, a nucleic sequence of which complementary nucleic acid molecule is a partial inversed-reversed sequence of the RNA, and transfecting host cells with the complementary nucleic acid molecule, thereby complementarily binding the RNA.

Still further in accordance with a preferred embodiment of the present invention the neutralizing includes immunologically neutralizing.

### BRIEF DESCRIPTION OF DRAWINGS

FIG. 1 is a simplified diagram illustrating a mode by which viral genes of a novel group of viral genes of the present invention, modulate expression of known host target genes;

FIG. 2 is a simplified block diagram illustrating a bioinformatic gene detection system capable of detecting genes of the novel group of viral genes of the present invention, which system is constructed and operative in accordance with a preferred embodiment of the present invention;

FIG. 3 is a simplified flowchart illustrating operation of a mechanism for training of a computer system to recognize the novel viral genes of the present invention, which mechanism is constructed and operative in accordance with a preferred embodiment of the present invention;

FIG. 4A is a simplified block diagram of a non-coding genomic sequence detector constructed and operative in accordance with a preferred embodiment of the present invention;

FIG. 4B is a simplified flowchart illustrating operation of a non-coding genomic sequence detector constructed and operative in accordance with a preferred embodiment of the present invention;

FIG. 5A is a simplified block diagram of a hairpin detector constructed and operative in accordance with a preferred embodiment of the present invention;

FIG. 5B is a simplified flowchart illustrating operation of a hairpin detector constructed and operative in accordance with a preferred embodiment of the present invention;

FIG. 6A is a simplified block diagram of a dicer-cut location detector constructed and operative in accordance with a preferred embodiment of the present invention;

FIG. 6B is a simplified flowchart illustrating training of a dicer-cut location detector constructed and operative in accordance with a preferred embodiment of the present invention;

FIG. 6C is a simplified flowchart illustrating operation of a dicer-cut location detector constructed and operative in accordance with a preferred embodiment of the present invention;

FIG. 7A is a simplified block diagram of a target-gene binding-site detector constructed and operative in accordance with a preferred embodiment of the present invention;

FIG. 7B is a simplified flowchart illustrating operation of a target-gene binding-site detector constructed and operative in accordance with a preferred embodiment of the present invention;

FIG. 8 is a simplified flowchart illustrating operation of a function & utility analyzer constructed and operative in accordance with a preferred embodiment of the present invention;

FIG. 9 is a simplified diagram describing a novel bioinformatically detected group of viral regulatory genes, referred to here as Viral Genomic Record (VGR) genes, each of which encodes an ‘operon-like’ cluster of novel miRNA-like viral genes, which in turn modulate expression of one or more host target genes;

FIG. 10 is a block diagram illustrating different utilities of novel viral genes and novel operon-like viral genes, both of the present invention;

FIGS. 11A and 11B are simplified diagrams, which when taken together illustrate a mode of gene therapy applicable to novel viral genes of the present invention;

FIG. 12 is a table summarizing laboratory validation results which validate efficacy of a bioinformatic gene detection system constructed and operative in accordance with a preferred embodiment of the present invention;

FIG. 13 is a picture of laboratory results validating the expression of 37 novel human genes detected by a bioinformatic gene detection engine constructed and operative in accordance with a preferred embodiment of the present invention, thereby validating the efficacy of the gene detection engine of the present invention;

FIG. 14A is a schematic representation of an ‘operon like’ cluster of novel human gene hairpin sequences detected bioinformatically by a bioinformatic gene detection engine constructed and operative in accordance with a preferred embodiment of the present invention, and non-GAM hairpin useful as negative controls thereto;

FIG. 14B is a schematic representation of secondary folding of hairpins of the operon-like cluster of FIG. 14A;

FIG. 14C is a picture of laboratory results demonstrating expression of novel human genes of FIGS. 14A and 14B, and lack of expression of the negative controls, thereby validating efficacy of bioinformatic detection of GAM genes and GR genes of the present invention, by a bioinformatic gene detection engine constructed and operative in accordance with a preferred embodiment of the present invention;

FIG. 15A is an annotated sequence of EST72223 comprising known human miRNA gene MIR98 and novel human gene GAM25, both detected by the gene detection system of the present invention; and

FIGS. 15B, 15C and 15D are pictures of laboratory results demonstrating laboratory confirmation of expression of known human gene MIR98 and of novel bioinformatically detected human gene GAM25 respectively, both of FIG. 15A, thus validating the bioinformatic gene detection system of the present invention.

FIG. 16 presents pictures of laboratory results demonstrating laboratory confirmation of ‘dicing’ of four novel bioinformatically detected HIV1 VGAMs into their corresponding mature genes, herein designated VGAM2032.2 (FIG. 16B), VGAM3249.1 (FIG. 16C), VGAM507.2 (FIG. 16D) and VGAM1016.2 (FIG. 16E).

FIG. 17 presents pictures of laboratory results demonstrating laboratory confirmation of expression of two novel bioinformatically detected Vaccinia VGAM precursors, herein designated VGAM224, and VGAM3184.

### DETAILED DESCRIPTION OF DRAWINGS

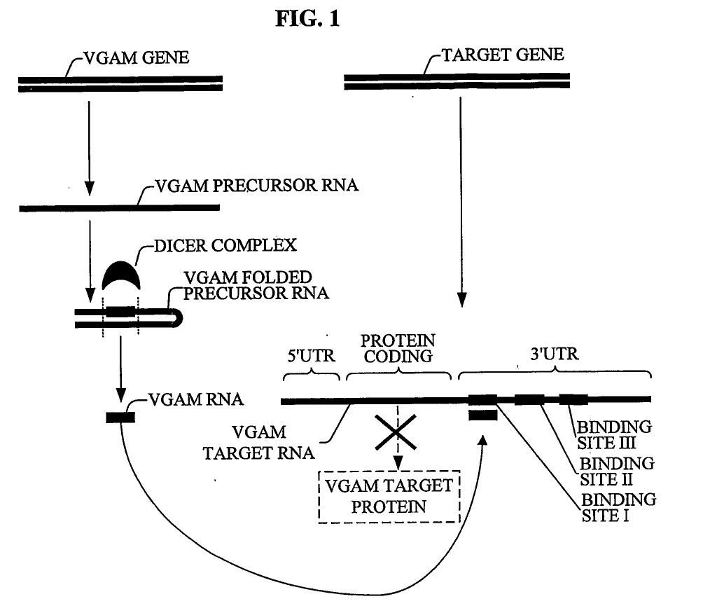

Reference is now made to FIG. 1, which is a simplified diagram describing each of a plurality of novel bioinformatically detected viral genes of the present invention, referred to here as Viral Genomic Address Messenger (VGAM) genes, which modulates expression of respective target genes thereof, the function and utility of which host target genes is known in the art.

VGAM is a novel bioinformatically detected regulatory, non protein coding, micro RNA (miRNA) viral gene. The method by which VGAM is detected is described hereinabove with reference to FIGS. 1-8.

VGAM GENE is gene contained in the virus genome and TARGET GENE is a human gene contained in the human genome.

VGAM GENE encodes a VGAM PRECURSOR RNA. Similar to other miRNA genes, and unlike most ordinary genes, VGAM PRECURSOR RNA does not encode a protein.

VGAM PRECURSOR RNA folds onto itself, forming VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurately or partially inversed reversed sequence of the nucleotide sequence of the second half thereof. By inversed reversed is meant a sequence which is reversed and wherein each nucleotide is replaced by a complementary nucleotide, as is well known in the art (e.g. ATGGC is the reverse complementary sequence of GCCAT).

An enzyme complex designated DICER COMPLEX, ‘dices’ the VGAM FOLDED PRECURSOR RNA into VGAM RNA, a single stranded ˜22 nt long RNA segment. As is known in the art, ‘dicing’ of a hairpin structured RNA precursor product into a short ˜22 nt RNA segment is catalyzed by an enzyme complex comprising an enzyme called Dicer together with other necessary proteins.

TARGET GENE encodes a corresponding messenger RNA, VGAM TARGET RNA. VGAM TARGET RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM RNA binds complementarily to one or more target binding sites located in untranslated regions of VGAM TARGET RNA. This complementary binding is due to the fact that the nucleotide sequence of VGAM RNA is a partial or accurate inversed reversed sequence of the nucleotide sequence of each of the host target binding sites. As an illustration, FIG. 1 shows three such target binding sites, designated BINDING SITE I, BINDING SITE II and BINDING SITE III respectively. It is appreciated that the number of host target binding sites shown in FIG. 1 is meant as an illustration only, and is not meant to be limiting VGAM RNA may have a different number of host target binding sites in untranslated regions of a VGAM TARGET RNA. It is further appreciated that while FIG. 1 depicts host target binding sites in the 3′UTR region, this is meant as an example only, these target binding sites may be located in the 3′UTR region, the 5′UTR region, or in both 3′UTR and 5′UTR regions.

The complementary binding of VGAM RNA to target binding sites on VGAM TARGET RNA, such as BINDING SITE I, BINDING SITE II and BINDING SITE III, inhibits translation of VGAM TARGET RNA into VGAM TARGET PROTEIN. VGAM TARGET PROTEIN is therefore outlined by a broken line.

It is appreciated that TARGET GENE in fact represents a plurality of VGAM target genes. The mRNA of each one of this plurality of VGAM target genes comprises one or more target binding sites, each having a nucleotide sequence which is at least partly complementary to VGAM RNA, and which when bound by VGAM RNA causes inhibition of translation of respective one or more VGAM host target proteins.

It is further appreciated by one skilled in the art that the mode of translational inhibition illustrated by FIG. 1 with specific reference to translational inhibition exerted by VGAM GENE on one or more TARGET GENE, is in fact common to other known miRNA genes, as is well known in the art.

Nucleotide sequences of each of a plurality of VGAM GENEs described by FIG. 1 and their respective genomic source and chromosomal location are further described hereinbelow with reference to Table 1, hereby incorporated by reference.

Nucleotide sequences of VGAM PRECURSOR RNA, and a schematic representation of a predicted secondary folding of VGAM FOLDED PRECURSOR RNA, of each of a plurality of VGAM GENEs described by FIG. 1 are further described hereinbelow with reference to Table 2, hereby incorporated by reference.

Nucleotide sequences of a ‘diced’ VGAM RNA of each of a plurality of VGAM GENEs described by FIG. 1 are further described hereinbelow with reference to Table 3, hereby incorporated by reference.

Nucleotide sequences of host target binding sites, such as BINDING SITE-I, BINDING SITE-II and BINDING SITE-III of FIG. 1, found on VGAM TARGET RNA, of each of a plurality of VGAM GENEs described by FIG. 1, and schematic representation of the complementarity of each of these host target binding sites to each of a plurality of VGAM RNA described by FIG. 1 are described hereinbelow with reference to Table 4, hereby incorporated by reference.

It is appreciated that specific functions and accordingly utilities of each of a plurality of VGAM GENEs described by FIG. 1 correlate with, and may be deduced from, the identity of the TARGET GENEs that each of said plurality of VGAM GENEs binds and inhibits, and the function of each of said TARGET GENEs, as elaborated hereinbelow with reference to Table 5, hereby incorporated by reference.

Studies establishing known functions of each of a plurality of TARGET GENEs of VGAM GENEs of FIG. 1, and correlation of said each of a plurality of TARGET GENEs to known diseases are listed in Table 6, and are hereby incorporated by reference.

The present invention discloses a novel group of genes, the VGAM genes, belonging to the miRNA genes group, and for which a specific complementary binding has been determined.

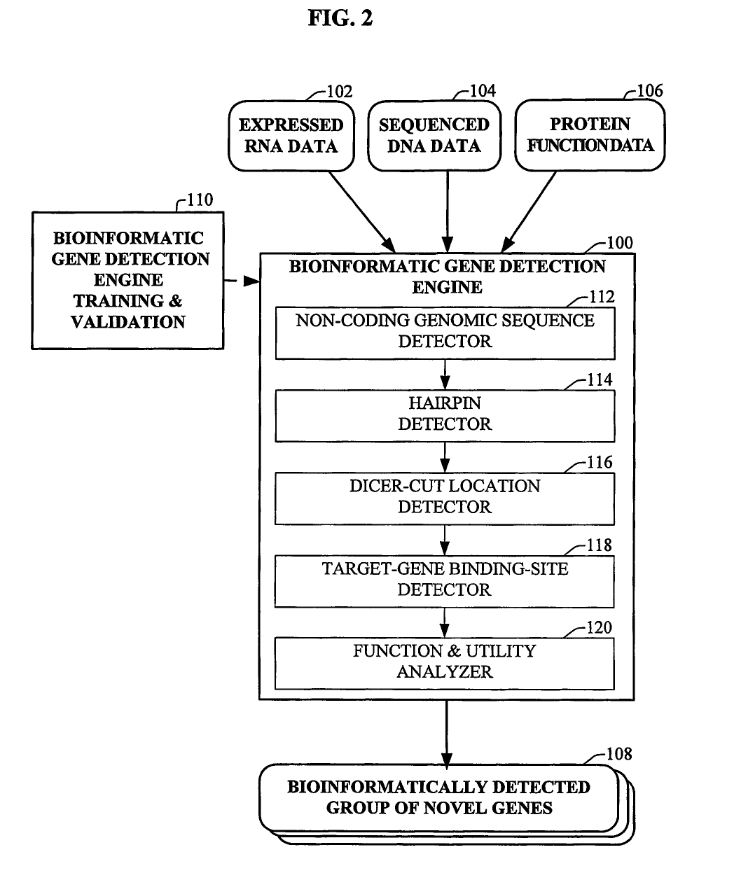

Reference is now made to FIG. 2 which is a simplified block diagram illustrating a bioinformatic gene detection system capable of detecting genes of the novel group of genes of the present invention, which system is constructed and operative in accordance with a preferred embodiment of the present invention.

A centerpiece of the present invention is a bioinformatic gene detection engine 100, which is a preferred implementation of a mechanism capable of bioinformatically detecting genes of the novel group of genes of the present invention.

The function of the bioinformatic gene detection engine 100 is as follows: it receives three types of input, expressed RNA data 102, sequenced DNA data 104, and protein function data 106, performs a complex process of analysis of this data as elaborated below, and based on this analysis produces output of a bioinformatically detected group of novel genes designated 108.

Expressed RNA data 102 comprises published expressed sequence tags (EST) data, published mRNA data, as well as other sources of published RNA data. Sequenced DNA data 104 comprises alphanumeric data describing sequenced genomic data, which preferably includes annotation data such as location of known protein coding regions relative to the sequenced data. Protein function data 106 comprises scientific publications reporting studies which elucidated physiological function known proteins, and their connection, involvement and possible utility in treatment and diagnosis of various diseases. Expressed RNA data 102 and sequenced DNA data 104 may preferably be obtained from data published by the National Center for Bioinformatics (NCBI) at the National Institute of Health (NIH) (Jenuth, 2000), as well as from various other published data sources. Protein function data 106 may preferably be obtained from any one of numerous relevant published data sources, such as the Online Mendelian Inherited Disease In Man (OMIM(™)) database developed by John Hopkins University, and also published by NCBI (2000).

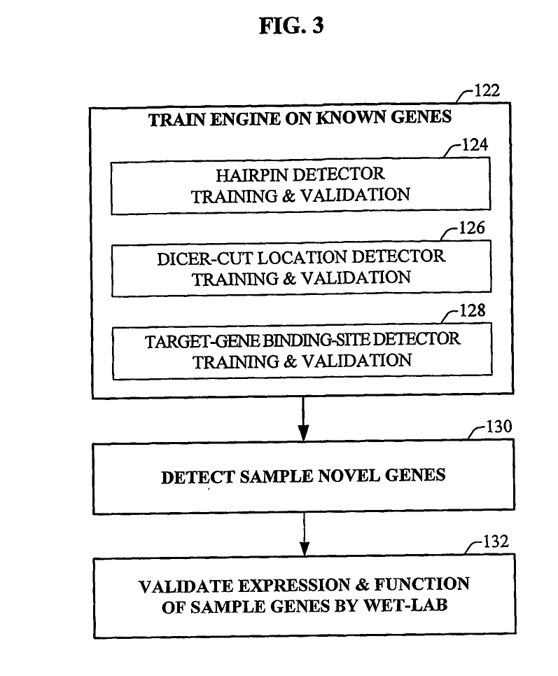

Prior to actual detection of bioinformatically detected novel genes 108 by the bioinformatic gene detection engine 100, a process of bioinformatic gene detection engine training & validation designated 110 takes place. This process uses the known miRNA genes as a training set (some 200 such genes have been found to date using biological laboratory means), to train the bioinformatic gene detection engine 100 to bioinformatically recognize miRNA-like genes, and their respective potential host target binding sites. Bioinformatic gene detection engine training & validation 110 is further described hereinbelow with reference to FIG. 3.

The bioinformatic gene detection engine 100 comprises several modules which are preferably activated sequentially, and are described as follows:

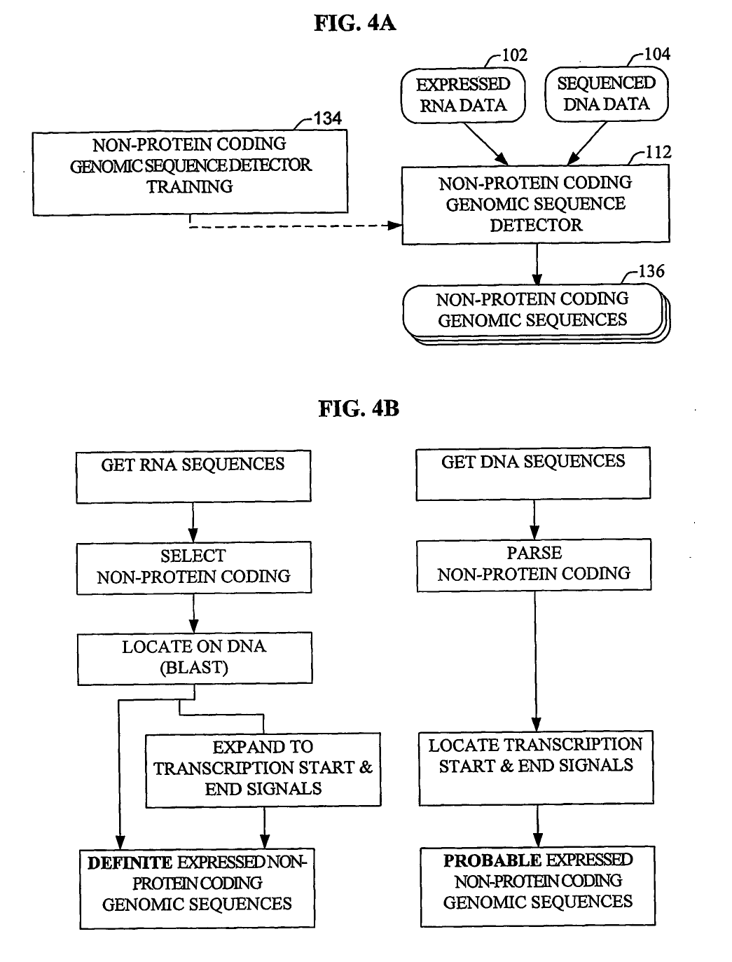

A non-coding genomic sequence detector 112 operative to bioinformatically detect non-protein coding genomic sequences. The non-coding genomic sequence detector 112 is further described herein below with reference to FIGS. 4A and 4B.

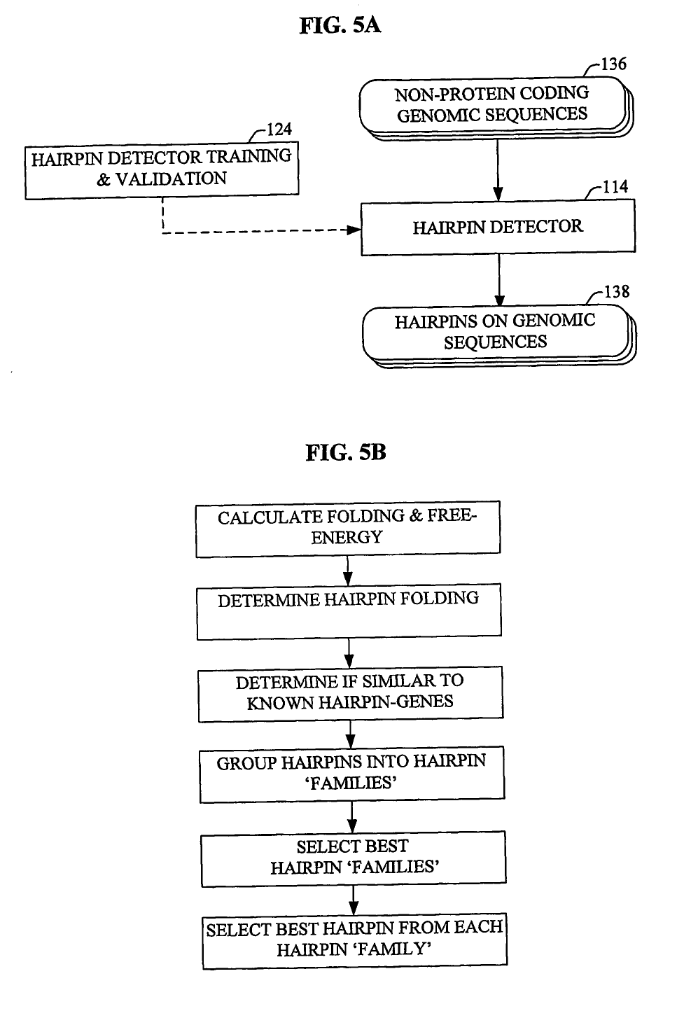

A hairpin detector 114 operative to bioinformatically detect genomic ‘hairpin-shaped’ sequences, similar to VGAM FOLDED PRECURSOR of FIG. 1. The hairpin detector 114 is further described herein below with reference to FIGS. 5A and 5B.

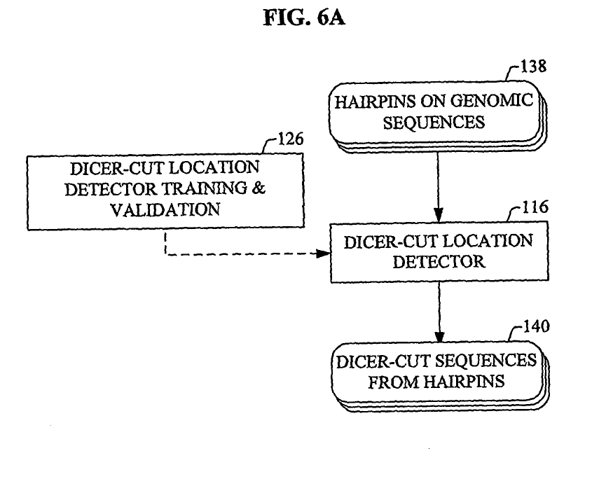

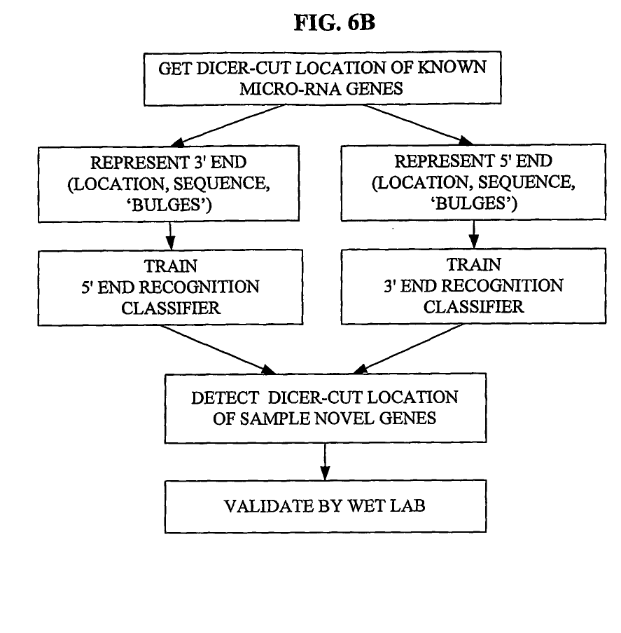

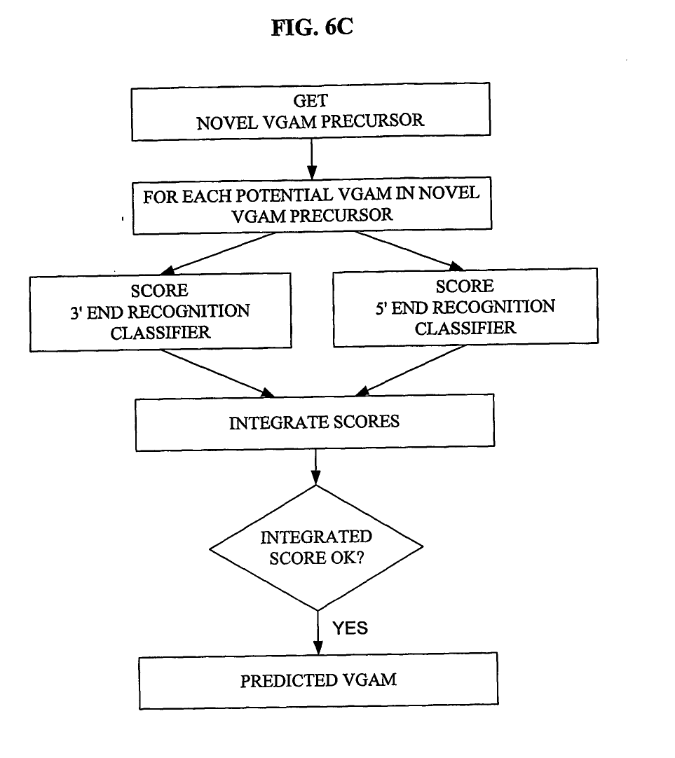

A dicer-cut cation detector 116 operative to bioinformatically detect the location on a hairpin shaped sequence which is enzymatically cut by DICER COMPLEX of FIG. 1. The dicer-cut location detector 116 is further described herein below with reference to FIG. 6A.

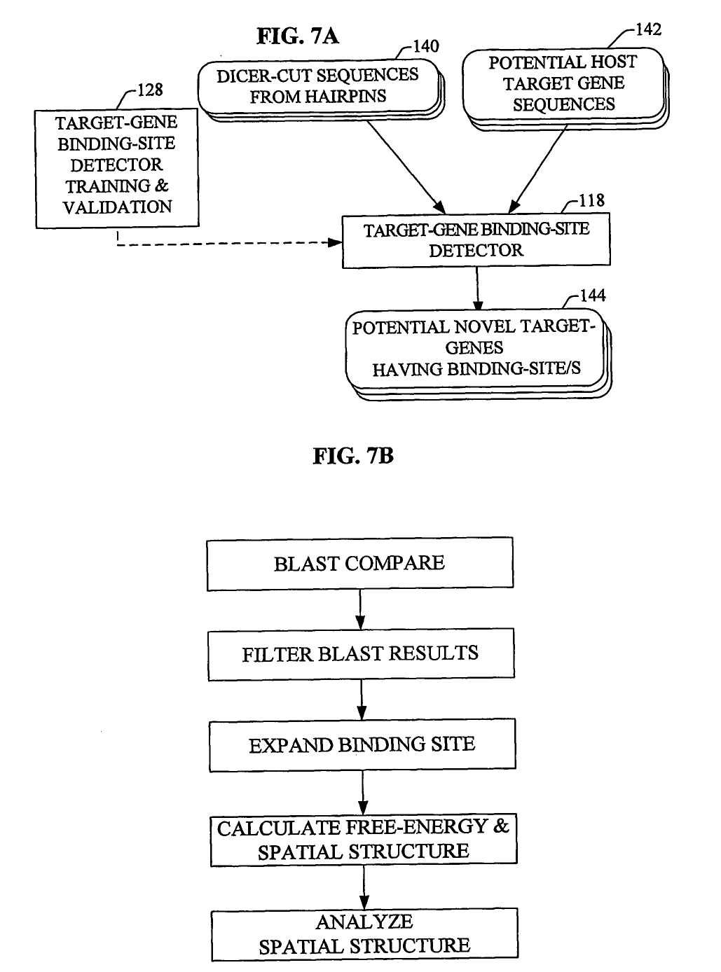

A target-gene binding-site detector 118 operative to bioinformatically detect host target genes having binding sites, the nucleotide sequence of which is partially complementary to that of a given genomic sequence, such as a sequence cut by DICER COMPLEX of FIG. 1. The target-gene binding-site detector 118 is further described hereinbelow with reference to FIGS. 7A and 7B.

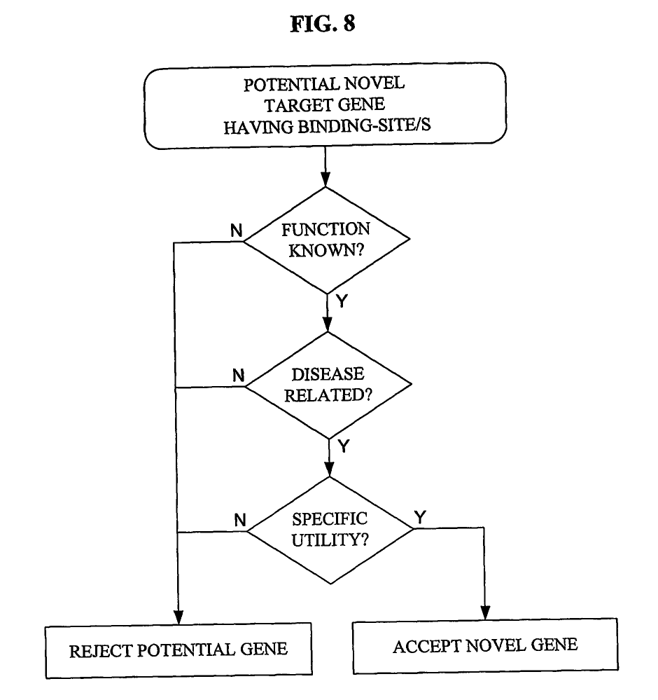

A function & utility analyzer 120 operative to analyze function and utility of target genes, in order to identify host target genes which have a significant clinical function and utility. The function & utility analyzer 120 is further described hereinbelow with reference to FIG. 8.

Hardware implementation of the bioinformatic gene detection engine 100 is important, since significant computing power is preferably required in order to perform the computation of bioinformatic gene detection engine 100 in reasonable time and cost. For example, it is estimated that a using a powerful 8-processor server (e.g. DELL POWEREDGE(™) 8450, 8 XEON(™) 550 MHz processors, 8 GB RAM), over 6 years (!) of computing time are required to detect all MIR genes in the human EST data, together with their respective binding sites. Various computer hardware and software configurations may be utilized in order to address this computation challenge, as is known in the art. A preferred embodiment of the present invention may preferably comprise a hardware configuration, comprising a cluster of one hundred PCs (PENTIUM(™) IV, 1.7 GHz, with 40 GB storage each), connected by Ethernet to 12 servers (2-CPU, XEON(™) 1.2-2.2 GHz, with ˜200 GB storage each), combined with an 8-processor server (8-CPU, Xeon 550 Mhz w/8 GB RAM) connected via 2 HBA fiber-channels to an EMC CLARIION(™) 100-disks, 3.6 Terabyte storage device. A preferred embodiment of the present invention may also preferably comprise a software configuration which utilizes a commercial database software program, such as MICROSOFT(™) SQL Server 2000. Using such preferred hardware and software configuration, may reduce computing time required to detect all MIR genes in the human EST data, and their respective binding sites, from 6 years to 45 days. It is appreciated that the above mentioned hardware configuration is not meant to be limiting, and is given as an illustration only. The present invention may be implemented in a wide variety of hardware and software configurations.

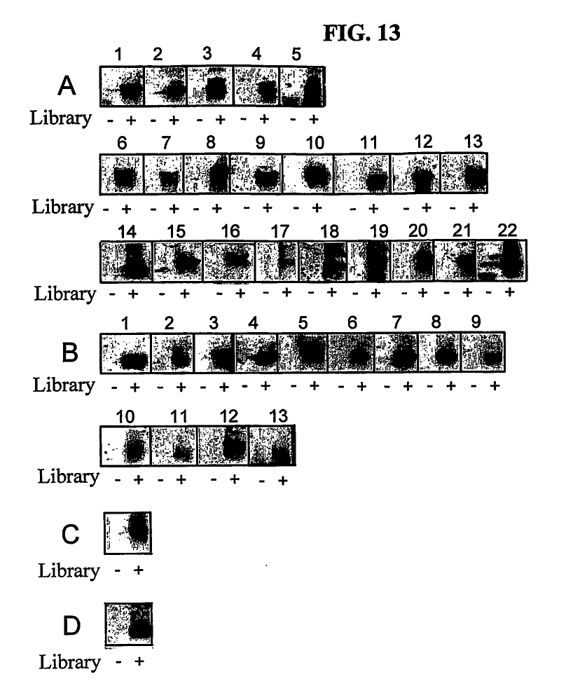

The present invention discloses 1560 novel genes of the VGAM group of genes, which have been detected bioinformatically, as described hereinbelow with reference to Table 1 through Table 6, and 205 novel genes of the GR group of genes, which have been detected bioinformatically, as described hereinbelow with reference to Table 7. Laboratory confirmation of 37 bioinformatically predicted genes of the human GAM and GR group of genes, and several bioinformatically predicted genes of the VGAM and VGR group of genes, is described hereinbelow with reference to FIGS. 13 through 17.

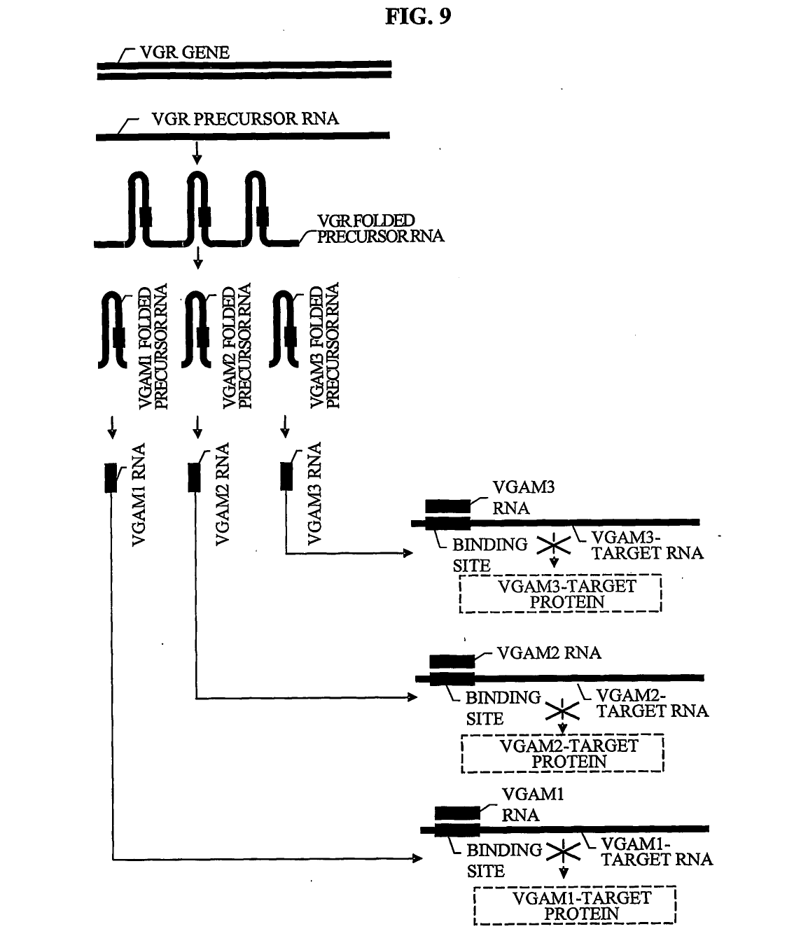

Reference is now made to FIG. 3 which is a simplified flowchart illustrating operation of a mechanism for training a computer system to recognize the novel genes of the present invention. This mechanism is a preferred implementation of the bioinformatic gene detection engine training & validation 110 described hereinabove with reference to FIG. 9.

BIOINFORMATIC GENE DETECTION ENGINE TRAINING & VALIDATION 110 of FIG. 2 begins by training the bioinformatic gene detection engine to recognize known miRNA genes, as designated by numeral 122. This training step comprises HAIRPIN DETECTOR TRAINING & VALIDATION 124, further described hereinbelow with reference to FIG. 5A, DICER-CUT LOCATION DETECTOR TRAINING & VALIDATION 126, further described hereinbelow with reference to FIGS. 6A and 6B, and TARGET-GENE BINDING-SITE DETECTOR TRAINING & VALIDATION 128, further described hereinbelow with reference to FIG. 7A.

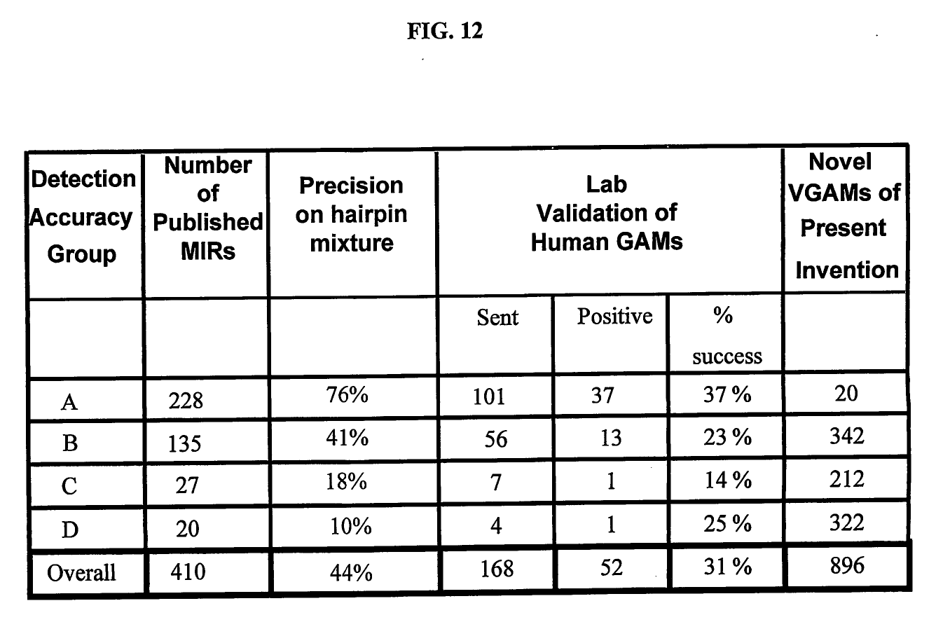

Next, the BIOINFORMATIC GENE DETECTION ENGINE 100 is used to bioinformatically detect sample novel genes, as designated by numeral 130. Examples of sample novel genes thus detected are described hereinbelow with reference to FIG. 12.

Finally, wet lab experiments are preferably conducted in order to validate expression and preferably function of the sample novel genes detected by the BIOINFORMATIC GENE DETECTION ENGINE 100 in the previous step. An example of wet-lab validation of the above mentioned sample novel gene bioinformatically detected by the system is described hereinbelow with reference to FIGS. 13 through 17.

Reference is now made to FIG. 4A which is a simplified block diagram of a preferred implementation of the NON-CODING GENOMIC SEQUENCE DETECTOR 112 described hereinabove with reference to FIG. 2. The NON-PROTEIN CODING GENOMIC SEQUENCE DETECTOR 112 of FIG. 2 preferably receives as input at least two types of published genomic data: EXPRESSED RNA DATA 102 and SEQUENCED DNA DATA 104. The EXPRESSED RNA DATA can include, among others, EST data, EST clusters data, EST genome alignment data and mRNA data. Sources for EXPRESSED RNA DATA 102 include NCBI dbEST, NCBI UniGene clusters and mapping data, and TIGR gene indices. SEQUENCED DNA DATA 104 includes both sequence data (FASTA format files), and features annotation (GenBank file format) mainly from NCBI database. After its initial training, indicated by numeral 134, and based on the above mentioned input data, the NON-PROTEIN CODING GENOMIC SEQUENCE DETECTOR 112 produces as output a plurality of NON-PROTEIN CODING GENOMIC SEQUENCES 136. Preferred operation of the NON-PROTEIN CODING GENOMIC SEQUENCE DETECTOR 112 is described hereinbelow with reference to FIG. 4B.

Reference is now made to FIG. 4B which is a simplified flowchart illustrating a preferred operation of the NON-CODING GENOMIC SEQUENCE DETECTOR 112 of FIG. 2. Detection of NON-PROTEIN CODING GENOMIC SEQUENCES 136, generally preferably progresses in one of the following two paths:

A first path for detecting NON-PROTEIN CODING GENOMIC SEQUENCES 136 begins by receiving a plurality of known RNA sequences, such as EST data. Each RNA sequence is first compared to all known protein-coding sequences, in order to select only those RNA sequences which are non-protein coding, i.e. intergenic or intronic. This can preferably be performed by sequence comparison of the RNA sequence to known protein coding sequences, using one of many alignment algorithms known in the art, such as BLAST. This sequence comparison to the DNA preferably also provides the localization of the RNA sequence on the DNA.

Alternatively, selection of non-protein coding RNA sequences and their localization to the DNA can be performed by using publicly available EST clusters data and genomic mapping databases, such as UNIGENE database published by NCBI or TIGR database, in order map expressed RNA sequences to DNA sequences encoding them, to find the right orientation of EST sequences, and to exclude ESTs which map to protein coding DNA regions, as is well known in the art. Public databases, such as TIGR, may also be used to map an EST to a cluster of ESTs, assumed to be expressed as one piece, and is known in the art as Tentative Human Consensus. Publicly available genome annotation databases, such as NCBI's GENBANK, may also be used to deduce expressed intronic sequences.

Optionally, an attempt may be made to ‘expand’ the non-protein RNA sequences thus found, by searching for transcription start and end signals, upstream and downstream of location of the RNA on the DNA respectively, as is well known in the art.

A second path for detecting non-protein coding genomic sequences starts by receiving DNA sequences. The DNA sequences are parsed into non protein coding sequences, based on published DNA annotation data, by extracting those DNA sequences which are between known protein coding sequences. Next, transcription start and end signals are sought. If such signals are found, and depending on their ‘strength’, probable expressed non-protein coding genomic sequences are yielded. Such approach is especially useful for identifying novel GAM genes which are found in proximity to other known miRNA genes, or other wet-lab validated GAM genes. Since, as described hereinbelow with reference to FIG. 9, VGAM genes are frequently found in clusters, therefore sequences near a known mRNA are more likely to contain novel genes. Optionally, sequence orthology, i.e. sequences conservation in an evolutionary related species, may be used to select genomic sequences having higher probability of containing expressed novel VGAM genes.

It is appreciated that in the present invention the bioinformatics gene detection engine 100 utilize the input genomic sequences, without filtering protein coding regions detected by the non-coding genomic sequence detector 112.

Reference is now made to FIG. 5A which is a simplified block diagram of a preferred implementation of the HAIRPIN DETECTOR 114 described hereinabove with reference to FIG. 2.

The goal of the HAIRPIN DETECTOR 114 is to detect ‘hairpin’ shaped genomic sequences, similar to those of known miRNA genes. As mentioned hereinabove with reference to FIG. 1, a ‘hairpin’ genomic sequence refers to a genomic sequence which ‘folds onto itself’ forming a hairpin like shape, due to the fact that nucleotide sequence of the first half of the nucleotide sequence is an accurate or partial complementary sequence of the nucleotide sequence of its second half.

The HAIRPIN DETECTOR 114 of FIG. 2 receives as input a plurality of NON-PROTEIN CODING GENOMIC SEQUENCES 136 of FIG. 4A. After a phase of HAIRPIN DETECTOR TRAINING & VALIDATION 124 of FIG. 3, the HAIRPIN DETECTOR 114 is operative to detect and output ‘hairpin shaped’ sequences, which are found in the input NON-PROTEIN CODING GENOMIC SEQUENCES 138. The hairpin shaped sequences detected by the HAIRPIN DETECTOR 114 are designated HAIRPINS ON GENOMIC SEQUENCES 138. Preferred operation of the HAIRPIN DETECTOR 114 is described hereinbelow with reference to FIG. 5B.

The phase of HAIRPIN DETECTOR TRAINING & VALIDATION 124 is an iterative process of applying the HAIRPIN DETECTOR 114 to known hairpin shaped miRNA genes, calibrating the HAIRPIN DETECTOR 114 such that it identifies the training set of known hairpins, as well as sequences which are similar thereto. In a preferred embodiment of the present invention, THE HAIRPIN DETECTOR TRAINING & VALIDATION 124 trains and validates each of the steps of operation of the HAIRPIN DETECTOR 114, which steps are described hereinbelow with reference to FIG. 5B.

The hairpin detector training and validation 124 preferably uses two sets of data: a training set of known miRNA genes, such as 440 miRNA genes of H. sapiens, M. musculus, C. elegans, C. Brigssae and D. Melanogaster, annotated in RFAM database (Griffiths-Jones 2003), and a large ‘background set’ of hairpins found in expressed non-protein coding genomic sequences, such as a set of 21,985 hairpins found in Tentative Human Concensus (THC) sequences in TIGR database. The ‘background set’ is expected to comprise some valid, previously undetected miRNA hairpins, and many hairpins which are not miRNA hairpins.

In order to validate the performance of the HAIRPIN DETECTOR 114, a validation method is preferably used, which validation method is a variation on the k-fold cross validation method (Mitchell, 1997). This preferred validation method is devised to better cope with the nature of the training set, which includes large families of similar and even identical miRNAs. The training set is preferably first divided into clusters of miRNAs such that any two miRNAs that belong to different clusters have an Edit Distance score (see Algorithms and Strings, Dan Gusfield, Cambridge University Press, 1997) of at least D=3, i.e. they differ by at least 3 editing operations. Next, the group of clusters is preferably divided into k sets. Then standard k-fold cross validation is preferably performed on this group of clusters, preferably using k=5, such that the members of each cluster are all in the training set or in the test set. It is appreciated that without the prior clustering, standard cross validation methods results in much higher performance of the predictors due to the redundancy of training examples, within the genome of a species and across genomes of different species.

In a preferred embodiment of the present invention, using the abovementioned validation method, the efficacy of the HAIRPIN DETECTOR 114 is indeed validated: for example, when a similarity threshold is chosen such that 90% of the published miRNA-precursor hairpins are successfully predicted, only 7.6% of the 21,985 background hairpins are predicted to be miRNA-precursors, some of which may indeed be previously unknown miRNA precursors.

Reference is now made to FIG. 5B which is a simplified flowchart illustrating a preferred operation of the HAIRPIN DETECTOR 114 of FIG. 2.

A hairpin structure is a secondary structure, resulting from the nucleotide sequence pattern: the nucleotide sequence of the first half of the hairpin is a partial or accurate inversed reversed sequence of the nucleotide sequence of the second half thereof. Various methodologies are known in the art for prediction of secondary and tertiary hairpin structures, based on given nucleotide sequences.

In a preferred embodiment of the present invention, the HAIRPIN DETECTOR 114 initially calculates possible secondary structure folding patterns of a given one of the non-protein coding genomic sequences 136 and the respective energy of each of these possible secondary folding patterns, preferably using a secondary structure folding algorithm based on free-energy minimization, such as the MFOLD algorithm (Mathews et al., 1999), as is well known in the art.

Next, the HAIRPIN DETECTOR 114 analyzes the results of the secondary structure folding, in order to determine the presence, and location of hairpin folding structures. A secondary structure folding algorithm, such as MFOLD algorithm, typically provides as output a listing of the base-pairing of the folded shape, i.e. a listing of each pair of connected nucleotides in the sequence. The goal of this second step is to asses this base-pairing listing, in order to determine if it describes a hairpin type bonding pattern. Preferably, each of the sequences that is determined to describe a hairpin structure is folded separately in order to determine its exact folding pattern and free-energy.

The HAIRPIN DETECTOR 114 then assess those hairpin structures found by the previous step, comparing them to hairpins of known miRNA genes, using various characteristic hairpin features such as length of the hairpin and of its loop, free-energy and thermodynamic stability, amount and type of mismatched nucleotides, existence of sequence repeat-elements. Only hairpins that bear statistically significant resemblance to the training set of known miRNA hairpins, according to the abovementioned parameters are accepted.

In a preferred embodiment of the present invention, similarity to the training set of known miRNA hairpins is determined using a ‘similarity score’ which is calculated using a weighted sum of terms, where each term is a function of one of the abovementioned hairpin features, and the parameters of each function are learned from the set of known hairpins, as described hereinabove with reference to hairpin detector training & validation 124. The weight of each term in the similarity score is optimized so as to achieve maximal separation between the distribution of similarity scores of hairpins which have been validated as miRNA-precursor hairpins, and the distribution of similarity scores of hairpins detected in the ‘background set’ mentioned hereinabove with reference to FIG. 5B, many of which are expected not to be miRNA-precursor hairpins.

In another preferred embodiment of the present invention, the abovementioned DETERMINE IF SIMILAR TO KNOWN HAIRPIN-GENES step may may preferably be split into two stages. The first stage is a permissive filter that implements a simplified scoring method, based on a subset of the hairpin features described hereinabove, such as minimal length and maximal free energy. The second stage is more stringent, and a full calculation of the weighted sum of terms described hereinabove is performed. This second stage may preferably be performed only on the subset of hairpins that survived prior filtering stages of the hairpin-detector 114.

Lastly, the HAIRPIN DETECTOR 114 attempts to select those hairpin structures which are as thermodynamically stable as the hairpins of known miRNA genes. This may preferably be achieved in various manners. A preferred embodiment of the present invention utilizes the following methodology preferably comprising three logical steps:

First, the HAIRPIN DETECTOR 114 attempts to group potential hairpins into ‘families’ of closely related hairpins. As is known in the art, a free-energy calculation algorithm, typically provides multiple ‘versions’ each describing a different possible secondary structure folding pattern for the given genomic sequence, and the free energy of such possible folding. The HAIRPIN DETECTOR 114 therefore preferably assesses all hairpins found in each of the ‘versions’, grouping hairpins which appear in different versions, but which share near identical locations into a common ‘family’ of hairpins. For example, all hairpins in different versions, the center of which hairpins is within 7 nucleotides of each other may preferably be grouped to a single ‘family’. Hairpins may also be grouped to a single ‘family’ if the sequences of one or more hairpins are identical to, or are subsequences of, the sequence of another hairpin.

Next, hairpin ‘families’ are assessed, in order to select only those families which represent hairpins that are as stable as those of known miRNA hairpins. Preferably only families which are represented in a majority of the secondary structure folding versions, such as at least in 65% or 80% or 100% of the secondary structure folding versions, are considered stable.

Finally, an attempt is made to select the most suitable hairpin from each selected family. For example, a hairpin which appears in more versions than other hairpins, and in versions the free-energy of which is lower, may be preferred.

In another preferred embodiment of the present invention, hairpins with homology to other species, and clusters of thermodynamically stable hairpin are further favored.

Reference is now made to FIG. 6A which is a simplified block diagram of a preferred implementation of the DICER-CUT LOCATION DETECTOR 116 described hereinabove with reference to FIG. 2.

The goal of the DICER-CUT LOCATION DETECTOR 116 is to detect the location in which DICER COMPLEX of FIG. 1, comprising the enzyme Dicer, would ‘dice’ the given hairpin sequence, similar to VGAM FOLDED PRECURSOR RNA, yielding VGAM RNA both of FIG. 1.

The DICER-CUT LOCATION DETECTOR 116 of FIG. 2 therefore receives as input a plurality of HAIRPINS ON GENOMIC SEQUENCES 138 of FIG. 5A, which were calculated by the previous step, and after a phase of DICER-CUT LOCATION DETECTOR TRAINING & VALIDATION 126, is operative to detect a respective plurality of DICER-CUT SEQUENCES FROM HAIRPINS 140, one for each hairpin.

In a preferred embodiment of the present invention, the DICER-CUT LOCATION DETECTOR 116 preferably uses standard machine learning techniques such as K nearest-neighbors, Bayesian networks and Support Vector Machines (SVM), trained on known dicer-cut locations of known miRNA genes in order to predict dicer-cut locations of novel VGAM genes. The DICER-CUT LOCATION DETECTOR TRAINING & VALIDATION 126 is further described hereinbelow with reference to FIG. 6B.

Reference is now made to FIG. 6B which is a simplified flowchart illustrating a preferred implementation of DICER-CUT LOCATION DETECTOR TRAINING & VALIDATION 126 of FIG. 3.

The general goal of the DICER-CUT LOCATION DETECTOR TRAINING & VALIDATION 126 is to analyze known hairpin shaped miRNA-precursors and their respective dicer-cut miRNA, in order to determine a common pattern to the dicer-cut location of known miRNA genes. Once such a common pattern is deduced, it may preferably be used by the DICER-CUT LOCATION DETECTOR 116, in detecting the predicted DICER-CUT SEQUENCES FROM HAIRPINS 140, from the respective HAIRPINS ON GENOMIC SEQUENCES 138, all of FIG. 6A.

First, the dicer-cut location of all known miRNA genes is obtained and studied, so as to train the DICER-CUT LOCATION DETECTOR 116: for each of the known miRNA, the location of the miRNA relative to its hairpin-shaped miRNA-precursor is noted.

The 5′ and 3′ ends of the dicer-cut location of each of the known miRNA genes is represented relative to the respective miRNA precursor hairpin, as well as to the nucleotides in each location along the hairpin. Frequency and identity of nucleotides and of nucleotide-pairing, and position of nucleotides and nucleotide pairing relative to the dicer-cut location in the known miRNA precursor hairpins is analyzed and modeled. In a preferred embodiment of the present invention, features learned from published miRNAs include: distance from hairpin's loop, nucleotide content, positional distribution of nucleotides and mismatched-nucleotides, and symmetry of mismatched-nucleotides.

Different techniques are well known in the art of machine learning for analysis of existing pattern from a given ‘training set’ of examples, which techniques are then capable, to a certain degree, to detect similar patterns in other, previously unseen examples. Such machine learning techniques include, but are not limited to neural networks, Bayesian networks, Support Vector Machines (SVM), Genetic Algorithms, Markovian modeling, Maximum Liklyhood modeling, Nearest Neighbor algorithms, Decision trees and other techniques, as is well known in the art.

The DICER-CUT LOCATION DETECTOR 116 preferably uses such standard machine learning techniques to predict either the 5′ end or both the 5′ and 3′ ends of the miRNA excised, or ‘diced’ by the Dicer enzyme from the miRNA hairpin shaped precursor, based on known pairs of miRNA-precursors and their respective resulting miRNAs. The nucleotide sequences of 440 published miRNA and their corresponding hairpin precursors are preferably used for training and evaluation of the dicer-cut location detector module.

Using the abovementioned training set, machine learning predictors, such as a Support Vector Machine (SVM) predictor, are implemented, which predictors test every possible nucleotide on a hairpin as a candidate for being the 5′ end or the 3′ end of a miRNA. Other machine learning predictors include predictors based on Nearest Neighbor, Bayesian modeling, and K-nearest-neighbor algorithms. The training set of the published miRNA precursor sequences is preferably used for training multiple separate classifiers or predictors, each of which produces a model for the 5′ or 3′ end of a miRNA relative to its hairpin precursor. The models take into account various miRNA properties such as the distance of the respective (3′ or 5′) end of the miRNA from the hairpin's loop, the nucleotides at its vicinity and the local ‘bulge’ (i.e. base-pair mismatch) structure.

Performance of the resulting predictors, evaluated on the abovementioned validation set of 440 published miRNAs using k-fold cross validation (Mitchell, 1997) with k=3, is found to be as follows: in 70% of known miRNAs 5′-end location is correctly determined by an SVM predictor within up to 2 nucleotides; a Nearest Neighbor (EDIT DISTANCE) predictor achieves 53% accuracy ( 233/440); a Two-Phased predictor that uses Baysian modeling (TWO PHASED) achieves 79% accuracy ( 348/440), when only the first phase is used, and 63% ( 277/440) when both phases are used; a K-nearest-neighbor predictor (FIRST-K) achieves 61% accuracy ( 268/440). The accuracies of all predictors are considerably higher on top scoring subsets of published miRNA.

Finally, in order to validate the efficacy and accuracy of the dicer-cut location detector 116, a sample of novel genes detected thereby is preferably selected, and validated by wet lab. Laboratory results validating the efficacy of the dicer-cut location detector 116 are described hereinbelow with reference to FIGS. 12 through 15D.

Reference is now made to FIG. 6C which is a simplified flowchart illustrating operation of DICER-CUT LOCATION DETECTOR 116 of FIG. 2, constructed and operative in accordance with a preferred embodiment of the present invention.

The DICER CUT LOCATION DETECTOR 116 is a machine learning computer program module, which is trained on recognizing dicer-cut location of known miRNA genes, and based on this training, is operable to detect dicer cut location of novel VGAM FOLDED PRECURSOR RNA. In a preferred embodiment of the present invention, the dicer-cut location module preferably utilizes machine learning algorithms, such as Support Vector Machine (SVM), Bayesian modeling, Nearest Neighbors, and K-nearest-neighbor, as is well known in the art.

When assessing a novel VGAM precursor, all 19-24 nucleotide long segments comprised in the VGAM precursor are initially considered as ‘potential VGAMs’, since the dicer-cut location is initially unknown.

For each such potential VGAM, its 5′ end, or its 5′ and 3′ ends are scored by two or more recognition classifiers or predictors.

In a preferred embodiment of the present invention, the DICER-CUT LOCATION DETECTOR 116 preferably uses a Support Vector Machine predictor trained on features such as distance from hairpin's loop, nucleotide content, positional distribution of nucleotides and mismatched-nucleotides, and symmetry of mismatched-nucleotides.

In another preferred embodiment of the present invention, the DICER-CUT LOCATION DETECTOR 116 preferably uses an ‘EDIT DISTANCE’ predictor, which seeks sequences that are similar to those of published miRNAs, utilizing the Nearest Neighbor algorithm, where the similarity metric between two sequences is a variant of the edit distance algorithm (Algorithms and Strings, Dan Gusfield, Cambridge University Press, 1997). This predictor is based on the observation that miRNAs tend to form clusters (Dostie, 2003), the members of which show marked sequence similarity to each other.

In yet another preferred embodiment of the present invention, the DICER-CUT LOCATION DETECTOR 116 preferably uses a ‘TWO PHASED’ predictor, which predicts the dicer-cut location in two distinct phases: (a) selecting the double-stranded segment of the hairpin comprising the miRNA by naïve Bayesian modeling (Mitchell, 1997), and (b) detecting which strand contains the miRNA by either naïve or by K-nearest-neighbor modeling. The latter is a variant of the ‘FIRST-K’ predictor described herein below, with parameters optimized for this specific task. The ‘TWO PHASED’ predictor may be operated in two modes: either utilizing only the first phase and thereby producing two alternative dicer-cut location predictions, or utilizing both phases and thereby producing only one final dicer-cut location.

In still another preferred embodiment of the present invention, the DICER-CUT LOCATION DETECTOR 116 preferably uses a ‘FIRST-K’ predictor, which utilizes the K-nearest-neighbor algorithm. The similarity metric between any two sequences is 1−E/L, where L is a parameter, preferably 8-10 and E is the edit distance between the two sequences, taking into account only the first L nucleotides of each sequence. If the K-nearest-neighbor scores of two or more locations on the hairpin are not significantly different, these locations are further ranked by a Bayesian model, similar to the one described hereinabove.

Scores of two or more of the abovementioned classifiers or predictors are integrated, yielding an integrated score for each ‘potential VGAM’. As an example, FIG. 13C illustrates integration of scores from two classifiers, a 3′ end recognition classifier and a 5′ end recognition classifier, the scores of which are integrated to yield an integrated score. In a preferred embodiment of the present invention, INTEGRATED SCORE of 13C preferably implements a ‘best-of-breed’ approach, accepting only ‘potential VGAMs’ that score highly on one of the above mentioned EDIT DISTANCE’, or ‘TWO-PHASED’ predictors. In this context, ‘high scores’ means scores which have been demonstrated to have low false positive value when scoring known miRNAs.

The INTEGRATED SCORE is then evaluated as follows: (a) the ‘potential VGAM’ having the highest score is taken to be the most probable VGAM, and (b) if the integrated score of this ‘potential VGAM’ is higher than a pre-defined threshold, then the potential VGAM is accepted as the PREDICTED VGAM.

Reference is now made to FIG. 7A which is a simplified block diagram of a preferred implementation of the TARGET-GENE BINDING-SITE DETECTOR 118 described hereinabove with reference to FIG. 2. The goal of the TARGET-GENE BINDING-SITE DETECTOR 118 is to detect a BINDING SITE of FIG. 1, including binding sites located in untranslated regions of the RNA of a known gene, the nucleotide sequence of which BINDING SITE is a partial or accurate inversed reversed sequence to that of a VGAM RNA of FIG. 1, thereby determining that the above mentioned known gene is a host target gene of VGAM of FIG. 1.

The TARGET-GENE BINDING-SITE DETECTOR 118 of FIG. 2 therefore receives as input a plurality of DICER-CUT SEQUENCES FROM HAIRPINS 140 of FIG. 6A which were calculated by the previous step, and a plurality of POTENTIAL HOST TARGET GENE SEQUENCES 142 which derive from SEQUENCED DNA DATA 104 of FIG. 2, and after a phase of TARGET-GENE BINDING-SITE DETECTOR TRAINING & VALIDATION 128 is operative to detect a plurality of POTENTIAL NOVEL TARGET-GENES HAVING BINDING SITE/S 144 the nucleotide sequence of which is a partial or accurate inversed reversed sequence to that of each of the plurality of DICER-CUT SEQUENCES FROM HAIRPINS 140. Preferred operation of the TARGET-GENE BINDING-SITE DETECTOR 118 is further described hereinbelow with reference to FIG. 7B.

Reference is now made to FIG. 7B which is a simplified flowchart illustrating a preferred operation of the target-gene binding-site detector 118 of FIG. 2.

In a preferred embodiment of the present invention, the target-gene binding-site detector 118 first uses a sequence comparison algorithm such as BLAST in order to compare the nucleotide sequence of each of the plurality of dicer-cut sequences from hairpins 140, to the potential host target gene sequences 142, such a untranslated regions of known mRNAs, in order to find crude potential matches. Alternatively, the sequence comparison may preferably be performed using a sequence match search tool that is essentially a variant of the EDIT DISTANCE algorithm described hereinabove with reference to FIG. 6C, and the Nearest Neighbor algorithm (Mitchell, 1997).

Results of the sequence comparison, performed by BLAST or other algorithms such as EDIT DISTANCE, are then filtered, preferably utilizing BLAST or EDIT DISTANCE score, to results which are similar to those of known binding sites (e.g. binding sites of miRNA genes Lin-4 and Let-7 to target genes Lin-14, Lin-41, Lin 28 etc.). Next the binding site is expanded, checking if nucleotide sequenced immediately adjacent to the binding site found by the sequence comparison algorithm (e.g. BLAST or EDIT DISTANCE), may improve the match. Suitable binding sites, then are computed for free-energy and spatial structure. The results are analyzed, accepting only those binding sites, which have free-energy and spatial structure similar to that of known binding sites. Since known binding sites of known miRNA genes frequently have multiple adjacent binding sites on the same target RNA, accordingly binding sites which are clustered are strongly preferred. Binding sites found in evolutionarily conserved sequences may preferably also be preferred.

For each candidate binding site a score, Binding Site Prediction Accuracy, is calculated which estimates their similarity of its binding to that of known binding sites. This score is based on VGAM-binding site folding features including, but not limited to the free-energy, the total number and distribution of base pairs, the total number and distribution of unpaired nucleotides.

In another preferred embodiment of the present invention binding sites are searched by a reversed process: sequences of K (preferably 22) nucleotides of the untranslated regions of the target gene are assessed as potential binding sites. A sequence comparison algorithm, such as BLAST or EDIT DISTANCE, is then used to search for partially or accurately complementary sequences elsewhere in the genome, which complementary sequences are found in known miRNA genes or computationally predicted VGAM genes. Only complementary sequences, the complementarity of which withstands the spatial structure and free energy analysis requirements described above are accepted. Clustered binding sites are strongly favored, as are potential binding sites and potential VGAM genes which occur in evolutionarily conserved genomic sequences.

Host target binding sites, identified by the TARGET-GENE BINDING-SITE DETECTOR 118, are divided into 4 groups: p) comprises binding sites that are exactly complementary to the predicted VGAM. a) b) and c) comprise binding sites that are not exactly complementary to the predicted VGAM: a) has binding sites with 0.9<Binding Site Prediction Accuracy<=1; b) has binding sites with 0.8<Binding Site Prediction Accuracy<=0.9; c) has binding sites with 0.7<Binding Site Prediction Accuracy<=0.8. The average number of mismatching nucleotides in the alignment of predicted VGAM and target binding site is smallest in category p) and largest in category c).

In a preferred embodiment of the current invention a ranking of VGAM to host target gene binding is performed by calculating a score, Target Accuracy. This score is the dominant group identifier of all binding sites of a specific VGAM to a specific host target gene UTR, where ‘a’ dominates ‘b’ and ‘b’ dominates ‘c’.

In yet another preferred embodiment of the current invention a ranking of VGAM to host target gene binding is performed directly from the set of Binding Site Prediction Accuracies corresponding to all the binding sites of a specific VGAM to a specific host target gene UTR. This set of Accuracies is sorted in descending order. The final Target Accuracy is a sum of two terms: the first is a weighted sum of the sorted Accuracies where the weights are exponentially decreasing as a function of the rank. The second term is a monotonously increasing function of the density of binding sites at the host target gene UTR.

Host target binding genes, identified by the TARGET-GENE BINDING-SITE DETECTOR 118, are divided into 4 groups according to their target binding genes: A) 0.75<Target Accuracy<=1; B) 0.65<Target Accuracy<=0.75; C) 0.5<Target Accuracy<=0.65; D) 0.3<Target Accuracy<=0.5

Reference is now made to FIG. 8 which is a simplified flowchart illustrating a preferred operation of the function & utility analyzer 120 described hereinabove with reference to FIG. 2. The goal of the function & utility analyzer 120 is to determine if a potential host target gene is in fact a valid clinically useful target gene. Since a potential novel VGAM gene binding a binding site in the UTR of a host target gene is understood to inhibit expression of that target gene, and if that host target gene is shown to have a valid clinical utility, then in such a case it follows that the potential novel viral gene itself also has a valid useful function—which is the opposite of that of the host target gene.

The function & utility analyzer 120 preferably receives as input a plurality of potential novel host target genes having binding-site/s 144, generated by the target-gene binding-site detector 118, both of FIG. 7A. Each potential viral gene, is evaluated as follows:

First, the system checks to see if the function of the potential host target gene is scientifically well established. Preferably, this can be achieved bioinformatically by searching various published data sources presenting information on known function of proteins. Many such data sources exist and are published as is well known in the art.

Next, for those host target genes the function of which is scientifically known and is well documented, the system then checks if scientific research data exists which links them to known diseases. For example, a preferred embodiment of the present invention utilizes the OMIM(™) database published by NCBI, which summarizes research publications relating to genes which have been shown to be associated with diseases.

Finally, the specific possible utility of the host target gene is evaluated. While this process too may be facilitated by bioinformatic means, it might require manual evaluation of published scientific research regarding the host target gene, in order to determine the utility of the host target gene to the diagnosis and or treatment of specific disease. Only potential novel viral genes, the host target-genes of which have passed all three examinations, are accepted as novel viral genes.

Reference is now made to FIG. 9, which is a simplified diagram describing each of a plurality of novel bioinformatically detected regulatory genes, referred to here as Viral Genomic Record (VGR) genes, which encodes an ‘operon-like’ cluster of novel micro RNA-like viral genes, each of which in turn modulates expression of at least one host target gene, the function and utility of which at least one host target gene is known in the art.

VGR GENE is a novel bioinformatically detected regulatory, non protein coding, RNA viral gene. The method by which VGR GENE was detected is described hereinabove with reference to FIGS. 1-9.

VGR GENE encodes VGR PRECURSOR RNA, an RNA molecule, typically several hundred nucleotides long.

VGR PRECURSOR RNA folds spatially, forming VGR FOLDED PRECURSOR RNA. It is appreciated that VGR FOLDED PRECURSOR RNA comprises a plurality of what is known in the art as ‘hairpin’ structures. These ‘hairpin’ structures are due to the fact that the nucleotide sequence of VGR PRECURSOR RNA comprises a plurality of segments, the first half of each such segment having a nucleotide sequence which is at least a partial or accurate inversed reversed sequence of the second half thereof, as is well known in the art.

VGR FOLDED PRECURSOR RNA is naturally processed by cellular enzymatic activity into separate VGAM precursor RNAs, herein schematically represented by VGAM1 FOLDED PRECURSOR RNA through VGAM3 FOLDED PRECURSOR RNA, each of which VGAM precursor RNAs being a hairpin shaped RNA segment, corresponding to VGAM FOLDED PRECURSOR RNA of FIG. 1.

The above mentioned VGAM precursor RNAs are diced by DICER COMPLEX of FIG. 1, yielding respective short RNA segments of about 22 nucleotides in length, schematically represented by VGAM1 RNA through VGAM3 RNA, each of which VGAM RNAs corresponding to VGAM RNA of FIG. 1.

VGAM1 RNA, VGAM2 RNA and VGAM3 RNA, each bind complementarily to binding sites located in untranslated regions of respective target genes, designated VGAM1-TARGET RNA, VGAM2-TARGET RNA and VGAM3-TARGET RNA, respectively, which target binding site corresponds to a target binding site such as BINDING SITE I, BINDING SITE II or BINDING SITE III of FIG. 1. This binding inhibits translation of the respective target proteins designated VGAM1-TARGET PROTEIN, VGAM2-TARGET PROTEIN and VGAM3-TARGET PROTEIN respectively.

It is appreciated that specific functions, and accordingly utilities, of each VGR GENE of the present invention, correlates with, and may be deduced from, the identity of the target genes, which are inhibited by VGAM RNAs comprised in the ‘operon-like’ cluster of said VGR GENE, schematically represented by VGAM1 TARGET PROTEIN through VGAM3 TARGET PROTEIN.

A listing of VGAM GENEs comprised in each of a plurality of VGR GENEs of FIG. 9 is provided in Table 7, hereby incorporated by reference. Nucleotide sequences of each said GAM GENEs and their respective genomic source and chromosomal location are further described hereinbelow with reference to Table 1, hereby incorporated by reference. TARGET GENEs of each of said GAM GENEs are elaborated hereinbelow with reference to Table 4, hereby incorporated by reference. The functions of each of said TARGET GENEs and their association with various diseases, and accordingly the utilities of said each of GAM GENEs, and hence the functions and utilities of each of said VGR GENEs of FIG. 9 is elaborated hereinbelow with reference to Table 5, hereby incorporated by reference. Studies establishing known functions of each of said TARGET GENEs, and correlation of each of said TARGET GENEs to known diseases are listed in Table 6, and are hereby incorporated by reference.

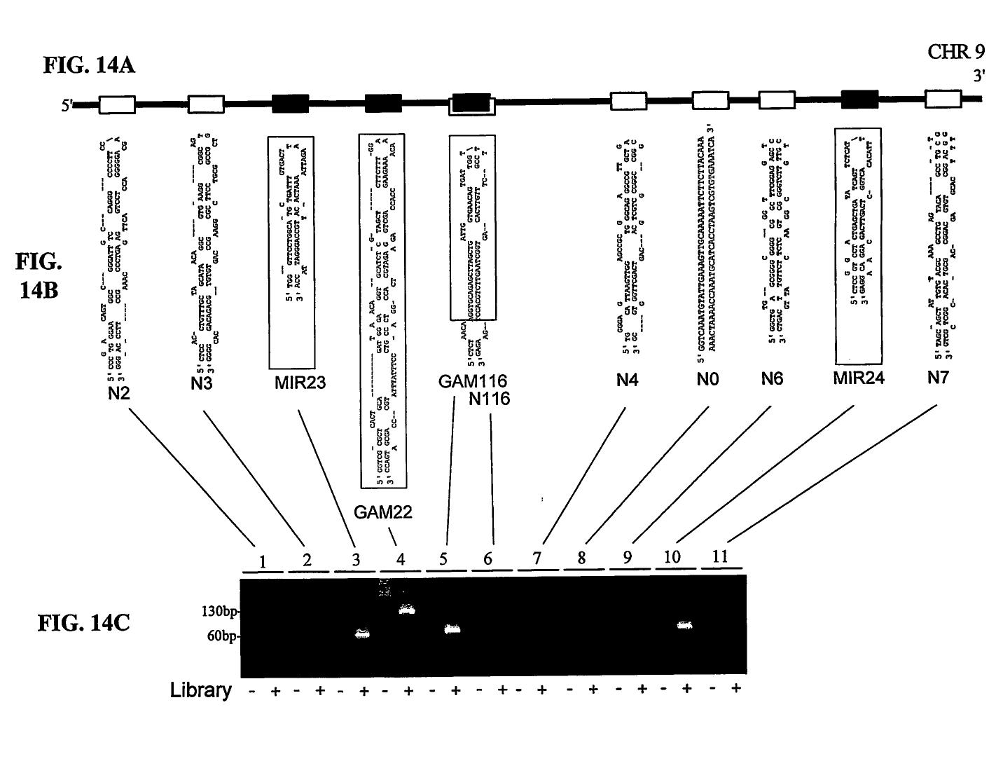

The present invention discloses 205 novel genes of the VGR group of genes, which have been detected bioinformatically, as elaborated hereinbelow with reference to Table 7. Laboratory confirmation of 2 genes of the GR group of genes is described hereinbelow with reference to FIGS. 14 through 17.

In summary, the current invention discloses a very large number of novel VGR genes, each of which encodes a plurality of VGAM genes, which in turn may modulate expression of a plurality of host target proteins.

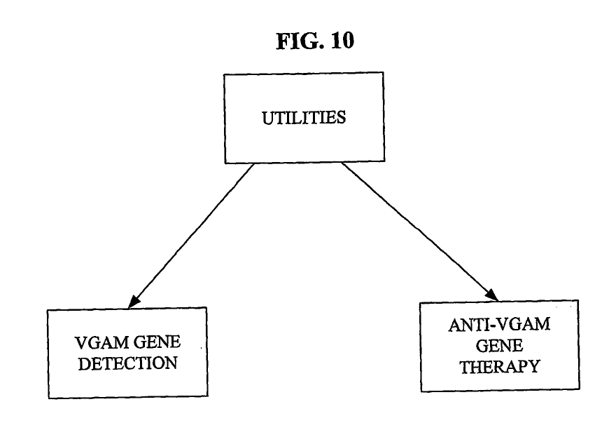

Reference is now made to FIG. 10 which is a block diagram illustrating different utilities of genes of the novel group of genes of the present invention referred to here as VGAM genes and VGR genes.

The present invention discloses a first plurality of novel viral genes referred to here as VGAM genes, and a second plurality of operon-like genes referred to here as VGR genes, each of the VGR genes encoding a plurality of VGAM genes. The present invention further discloses a very large number of known host target-genes, which are bound by, and the expression of which is modulated by each of the novel viral genes of the present invention. Published scientific data referenced by the present invention provides specific, substantial, and credible evidence that the above mentioned host target genes modulated by novel viral genes of the present invention, are associated with various diseases. Specific novel genes of the present invention, host target genes thereof and diseases associated therewith, are described hereinbelow with reference to Tables 1 through 7. It is therefore appreciated that a function of VGAM genes and VGR genes of the present invention is modulation of expression of host target genes related to known diseases, and that therefore utilities of novel genes of the present invention include diagnosis and treatment of the above mentioned diseases. FIG. 10 describes various types of diagnostic and therapeutic utilities of novel genes of the present invention.

A utility of novel genes of the present invention is detection of VGAM genes and of VGR genes. It is appreciated that since VGAM genes and VGR genes modulate expression of disease related host target genes, that detection of expression of VGAM genes in clinical scenarios associated with said diseases is a specific, substantial and credible utility. Diagnosis of novel viral genes of the present invention may preferably be implemented by RNA expression detection techniques, including but not limited to biochips, as is well known in the art. Diagnosis of expression of genes of the present invention may be useful for research purposes, in order to further understand the connection between the novel genes of the present invention and the above mentioned related diseases, for disease diagnosis and prevention purposes, and for monitoring disease progress.

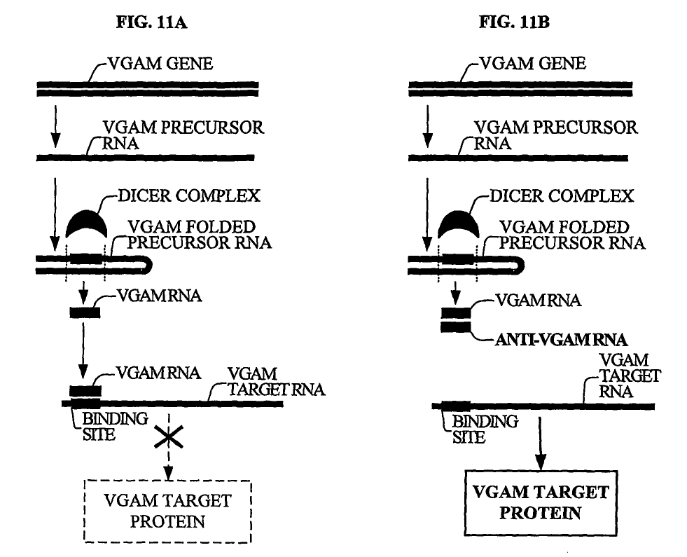

Another utility of novel viral genes of the present invention is anti-VGAM gene therapy, a mode of therapy which allows up regulation of a disease related host target-gene of a novel VGAM gene of the present invention, by lowering levels of the novel VGAM gene which naturally inhibits expression of that host target gene. This mode of therapy is particularly useful with respect to target genes which have been shown to be under-expressed in association with a specific disease. Anti-VGAM gene therapy is further discussed hereinbelow with reference to FIGS. 11A and 11B.

Reference is now made to FIGS. 11A and 11B, simplified diagrams which when taken together illustrate anti-VGAM gene therapy mentioned hereinabove with reference to FIG. 10. A utility of novel genes of the present invention is anti-VGAM gene therapy, a mode of therapy which allows up regulation of a disease related host target-gene of a novel VGAM gene of the present invention, by lowering levels of the novel VGAM gene which naturally inhibits expression of that host target gene. FIG. 11A shows a normal VGAM gene, inhibiting translation of a host target gene of VGAM gene, by binding to a BINDING SITE found in an untranslated region of VGAM TARGET RNA, as described hereinabove with reference to FIG. 1.

FIG. 11B shows an example of anti-VGAM gene therapy. ANTI-VGAM RNA is short artificial RNA molecule the sequence of which is an anti-sense of VGAM RNA. Anti-VGAM treatment comprises transfecting diseased cells with ANTI-VGAM RNA, or with a DNA encoding thereof. The ANTI-VGAM RNA binds the natural VGAM RNA, thereby preventing binding of natural VGAM RNA to its BINDING SITE. This prevents natural translation inhibition of VGAM TARGET RNA by VGAM RNA, thereby up regulating expression of VGAM TARGET PROTEIN.

It is appreciated that anti-VGAM gene therapy is particularly useful with respect to host target genes which have been shown to be under-expressed in association with a specific disease.

Furthermore, anti-VGAM therapy is particularly useful, since it may be used in situations in which technologies known in the art as RNAi and siRNA can not be utilized. As in known in the art, RNAi and siRNA are technologies which offer means for artificially inhibiting expression of a target protein, by artificially designed short RNA segments which bind complementarily to mRNA of said target protein. However, RNAi and siRNA can not be used to directly upregulate translation of target proteins.

Reference is now made to FIG. 12, which is a table summarizing laboratory validation results that validate efficacy of the bioinformatic gene detection engine 100 of FIG. 2. In order to assess efficacy of the bioinformatic gene detection engine 100, novel genes predicted thereby are preferably divided into 4 DETECTION ACCURACY GROUPS (first column), designated A through D, ranking VGAMS from the most probable VGAMs to the least probable VGAMs, using the scores of HAIRPIN DETECTOR 114 and DICER-CUT LOCATION DETECTOR 116 as follows:

Group A: score of the HAIRPIN-DETECTOR is above 0.7, the overall score of the two-phased predictor is above 0.55, and the score of the second phase of the two-phased predictor is above 0.75, or the score of the EDIT-DISTANCE predictor is equal or above 17. In this group, one miRNA is predicted for each hairpin. Group B: The score of the HAIRPIN-DETECTOR is above 0.5, the overall score of the two-phased predictor is above 0.55, and the hairpin is not in group A. Group C: The score of the HAIRPIN-DETECTOR is between 0.4 and 0.5, and the overall score of the two-phased predictor is above 0.55. Group D: The score of the HAIRPIN-DETECTOR is between 0.3 and 0.4, and the overall score of the two-phased predictor is above 0.55. In groups B, C and D, if the score of the second phase of the two-phased predictor is above 0.75, one miRNA is predicted for each hairpin, otherwise both sides of the double stranded window are given as output, and are examined in the lab or used for binding site search. The groups are mutually exclusive, i.e. in groups A, C and D all hairpins score less than 17 in the EDIT-DISTANCE predictor.

It is appreciated that the division into groups is not exhaustive: 410 of the 440 published hairpins (second column), and 896 of the 1560 novel VGAMs, belong to one of the groups. An indication of the real performance of the two phased predictor in the presence of background hairpins is given by the column ‘precision on hairpin mixture’ (third column). The precision on hairpin mixture is computed by mixing the published hairpins with background hairpins in a ratio of 1:4 and taking as a working assumption that they are hairpins not carrying a miRNA. This is a strict assumption, since some of these background hairpins may indeed contain miRNAs, while in this column they are all counted as failures

Sample novel bioinformatically predicted human genes, of each of these groups are sent to the laboratory for validation (fourth column), and the number (fifth column) and percent (sixth column) of successful validation of predicted human GAM genes is noted for each of the groups, as well as overall (bottom line). The number of novel VGAM genes explicitly specified by present invention belonging to each of the four groups is noted (seventh column).

It is appreciated that the present invention comprises 896 novel VGAM genes, which fall into one of these four detection accuracy groups, and that the bioinformatic gene detection engine 100 of FIG. 2 is substantiated by a group of 52 novel human GAM genes validated by laboratory means, out of 168 human GAM genes which were tested in the lab, resulting in validation of an overall 31% accuracy. The top group demonstrated 37% accuracy. Pictures of test-results of specific human genes in the abovementioned four groups, as well as the methodology used for validating the expression of predicted genes is elaborated hereinbelow with reference to FIG. 13.

It is further appreciated that failure to detect a gene in the lab does not necessarily indicate a mistaken bioinformatic prediction. Rather, it may be due to technical sensitivity limitation of the lab test, or because the gene is not expressed in the tissue examined, or at the development phase tested.

It is still further appreciated that in general these findings are in agreement with the expected bioinformatic accuracy, as describe hereinabove with reference to FIG. 6B: assuming 80% accuracy of the hairpin detector 114 and 80% accuracy of the dicer-cut location detector 116 and 80% accuracy of the lab validation, this would result in 50% overall accuracy of the genes validated in the lab.

Reference is now made to FIG. 13 which is a picture of laboratory results validating the expression of 37 novel human genes detected by the bioinformatic gene detection engine 100, in the four detection accuracy groups A through D described hereinabove with reference to FIG. 12.

Each row in FIG. 13, designated A through D, correlates to a corresponding one of the four detection accuracy groups A-D, described hereinabove with reference to FIG. 12. In each row, pictures of several genes validated by hybridization of PCR-product southern-blots, are provided, each corresponding to a specific GAM gene, as elaborated hereinbelow. These PCR-product hybridization pictures are designated 1 through 22 in the A group, 1 through 13 in the B group, 1 in the C group, and 1 in the D group. In each PCR hybridization picture, 2 lanes are seen: the test lane, designated ‘+’ and the control lane, designated ‘−’. For convenience of viewing the results, all PCR-product hybridization pictures of FIG. 13 have been shrunk ×4 vertically. It is appreciated that for each of the tested genes, a clear hybridization band appears in the test (‘+’) lane, but not in the control (‘−’) lane.

Specifically, FIG. 13 shows pictures of PCR-product hybridization validation by southern-blot, the methodology of which is described hereinbelow, to the following novel human GAM genes (RosettaGenomics Ltd. Gene Nomenclature):

DETECTION ACCURACY GROUP A: (1) GAM8297.1; (2) GAM5346.1; (3) GAM281.1; (4) GAM8554.1; (5) GAM2071.1; (6) GAM7553.1; (7) GAM5385.1; (8) GAM5227.1; (9) GAM7809.1; (10) GAM1032.1; (11) GAM3431.1; (12) GAM7933.1; (13) GAM3298.1;(14) GAM116.1; (15) GAM3418.1 (later published by other researchers as MIR23); (16) GAM3499.1; (17) GAM3027.1; (18) GAM7080.1; (19) GAM895.1; and (20) GAM2608.1, (21) GAM20, and (22) GAM21.

DETECTION ACCURACY GROUP B: (1) GAM3770.1; (2) GAM1338.1; (3) GAM7957.1; (4) GAM391.1; (5) GAM 8678.1; (6) GAM2033.1; (7)GAM7776.1; (8) GAM8145.1; (9) GAM 633.1; (10) GAM19; (11) GAM8358.1; (12) GAM3229.1; and (13) GAM7052.1.

DETECTION ACCURACY GROUP C: GAM25.

DETECTION ACCURACY GROUP D: GAM7352.1.

In addition to the PCR detection, the following GAMs were cloned and sequenced: GAM1338.1, GAM7809.1, GAM116.1, GAM3418.1 (later published by other researchers as MIR23), GAM3499.1, GAM3027.1, GAM7080.1, and GAM21.

The PCR-product hybridization validation methodology used is briefly described as follows. In order to validate the expression of predicted novel GAM/VGAM genes, and assuming that these novel genes are probably expressed at low concentrations, a PCR product cloning approach was set up through the following strategy: two types of cDNA libraries designated “One tailed” and “Ligation” were prepared from frozen HeLa S100 extract (4c Biotech, Belgium) size fractionated RNA. Essentially, Total S100 RNA was prepared through an SDS-Proteinase K incubation followed by an acid Phenol-Chloroform purification and Isopropanol precipitation. Alternatively, total HeLa RNA was also used as starting material for these libraries.

Fractionation was done by loading up to 500 μg per YM100 Amicon Microcon column (Millipore) followed by a 500 g centrifugation for 40 minutes at 4° C. Flowthrough “YM100” RNA consisting of about ¼ of the total RNA was used for library preparation or fractionated further by loading onto a YM30 Amicon Microcon column (Millipore) followed by a 13,500 g centrifugation for 25 minutes at 4° C. Flowthrough “YM30” was used for library preparation as is and consists of less than 0.5% of total RNA. For the both the “ligation” and the “One-tailed” libraries RNA was dephosphorilated and ligated to an RNA (lowercase)-DNA (UPPERCASE) hybrid 5′-phosphorilated, 3′ idT blocked 3′-adapter (5′-P-uuuAACCGCATTCTC-idT-3′ Dharmacon # P-002045-01-05) (as elaborated in Elbashir et al 2001) resulting in ligation only of RNase III type cleavage products. 3′-Ligated RNA was excised and purified from a half 6%, half 13% polyacrylamide gel to remove excess adapter with a Nanosep 0.2 μM centrifugal device (Pall) according to instructions, and precipitated with glycogen and 3 volumes of Ethanol. Pellet was resuspended in a minimal volume of water. For the “ligation” library a DNA (UPPERCASE)-RNA (lowercase) hybrid 5′-adapter (5′-TACTAATACGACTCACTaaa-3′ Dharmacon # P-002046-01-05) was ligated to the 3′-adapted RNA, reverse transcribed with “EcoRI-RT”: (5′-GACTAGCTGGAATTCAAGGATGCGGTTAAA-3′), PCR amplified with two external primers essentially as in Elbashir et al 2001 except that primers were “EcoRI-RT” and “PstI Fwd” (5′-CAGCCAACGCTGCAGATACGACTCACTAAA-3′). This PCR product was used as a template for a second round of PCR with one hemispecific and one external primer or with two hemispecific primers.

For the “One tailed” library the 3′-Adapted RNA was annealed to 20 pmol primer “EcoRI RT” by heating to 70° C. and cooling 0.1° C./sec to 30° C. and then reverse transcribed with Superscript II RT (According to instructions, Invitrogen) in a 20 μl volume for 10 alternating 5 minute cycles of 37° C. and 45° C. Subsequently, RNA was digested with 1 μl 2M NaOH, 2 mM EDTA at 65° C. for 10 minutes. cDNA was loaded on a polyacrylamide gel, excised and gel-purified from excess primer as above (invisible, judged by primer run alongside) and resuspended in 13 μl of water. Purified cDNA was then oligo-dC tailed with 400 U of recombinant terminal transferase (Roche molecular biochemicals), 1 μl 100 μM dCTP, 1 μl 15 mM CoCl₂, and 4 μl reaction buffer, to a final volume of 20 μl for 15 minutes at 37° C. Reaction was stopped with 2 μl 0.2M EDTA and 15 μl 3M NaOAc pH 5.2. Volume was adjusted to 150 μl with water, Phenol:Bromochloropropane 10:1 extracted and subsequently precipitated with glycogen and 3 volumes of Ethanol. C-tailed cDNA was used as a template for PCR with the external primers “T3-PstBsg(G/I)₁₈” (5′-AATTAACCCTCACTAAAGGCTGCAGGTGCAGGIGGGIIGGGIIGGGIIGN-3′ where I stands for Inosine and N for any of the 4 possible deoxynucleotides), and with “EcoRI Nested” (5′-GGAATTCAAGGATGCGGTTA-3′). This PCR product was used as a template for a second round of PCR with one hemispecific and one external primer or with two hemispecific primers.

Hemispecific primers were constructed for each predicted GAM/VGAM by an in-house program designed to choose about half of the 5′ or 3′ sequence of the GAM/VGAM corresponding to a TM° of about 30°-34° C. constrained by an optimized 3′ clamp, appended to the cloning adapter sequence (for “One-tailed” libraries 5′-GGNNGGGNNG on the 5′ end of the GAM/VGAM, or TTTAACCGCATC-3′ on the 3′ end of the GAM/VGAM. For “Ligation” libraries the same 3′ adapter and 5′-CGACTCACTAAA on the 5′ end). Consequently, a fully complementary primer of a TM° higher than 60° C. was created covering only one half of the GAM/VGAM sequence permitting the unbiased elucidation by sequencing of the other half.

Confirmation of GAM/VGAM Sequence Authenticity of PCR Products:

SOUTHERN BLOT: PCR-product sequences were confirmed by southern blot (Southern EM. Biotechnology. 1992; 24:122-39. (1975)) and hybridization with DNA oligonucleotide probes synthesized against predicted GAMs/VGAMs. Gels were transferred onto a Biodyne PLUS 0.45 μm, (Pall) positively charged nylon membrane and UV cross-linked. Hybridization was performed overnight with DIG-labeled probes at 42° C. in DIG Easy-Hyb buffer (Roche). Membranes were washed twice with 2×SSC and 0.1% SDS for 10 min. at 42° C. and then washed twice with 0.5×SSC and 0.1% SDS for 5 min at 42° C. The membrane was then developed by using a DIG luminescent detection kit (Roche) using anti-DIG and CSPD reaction, according to the manufacturer's protocol. All probes were prepared according to the manufacturers (Roche Molecular Biochemicals) protocols: Digoxigenin (DIG) labeled antisense transcripts was prepared from purified PCR products using a DIG RNA labeling kit with T3 RNA polymerase. DIG labeled PCR was prepared by using a DIG PCR labeling kit. 3′-DIG-tailed oligo ssDNA antisense probes, containing DIG-dUTP and dATP at an average tail length of 50 nucleotides were prepared from 100 pmole oligonucleotides with the DIG Oligonucleotide Labeling Kit.

CLONING: PCR products were inserted into pGEM-T (Promega) or pTZ57 (MBI Fermentas), transformed into competent JM109 E. coli (Promega) and sown on LB-Amp plates with IPTG/Xgal. White and light-blue colonies were transferred to duplicate gridded plates, one of which was blotted onto a membrane (Biodyne Plus, Pall) for hybridization with DIG tailed oligo probes (according to instructions, Roche) corresponding to the expected GAM. Plasmid DNA from positive colonies was sequenced.

Reference is now made to FIG. 14A, which is a schematic representation of a novel human GR gene, herein designated GR12731 (RosettaGenomics Ltd. Gene Nomenclature), located on chromosome 9, comprising 2 known MIR genes—MIR23 MIR24, and 2 novel GAM genes, herein designated GAM22 and GAM116, all marked by solid black boxes. FIG. 14A also schematically illustrates 6 non-GAM hairpin sequences, and one non-hairpin sequence, all marked by white boxes, and serving as negative controls. By ‘non-GAM hairpin sequences’ is meant sequences of a similar length to known MIR PRECURSOR sequences, which form hairpin secondary folding pattern similar to MIR PRECURSOR hairpins, and yet which are assessed by the bioinformatic gene detection engine 100 not to be valid GAM PRECURSOR hairpins. It is appreciated that FIG. 14A is a simplified schematic representation, reflecting only the order in which the segments of interest appear relative to one another, and not a proportional distance between the segments.

Reference is now made to FIG. 14B, which is a schematic representation of secondary folding of each of the MIRs and GAMs of GR GR12731—MIR24, MIR23, GAM22 and GAM116, and of the negative control non-GAM hairpins, herein designated N2, N3, N116, N4, N6 and N7. N0 is a non-hairpin control, of a similar length to that of known MIR PRECURSOR hairpins. It is appreciated that the negative controls are situated adjacent to and in between real MIR and GAM genes, and demonstrates similar secondary folding patterns to that of known MIRs and GAMs.

Reference is now made to FIG. 14C, which is a picture of laboratory results of a PCR test upon a YM100 “ligation”-library, utilizing specific primer sets directly inside the boundaries of the hairpins. Due to the nature of the library the only PCR amplifiable products can result from RNaseIII type enzyme cleaved RNA, as expected for legitimate hairpin precursors presumed to be produced by DROSHA (Lee et al, Nature 425 415-419, 2003). FIG. 14C demonstrates expression of hairpin precursors of known MIR genes—MIR23 and MIR24, and of novel bioinformatically detected GAM22 and GAM116 genes predicted bioinformatically by a system constructed and operative in accordance with a preferred embodiment of the present invention. FIG. 14C also shows that none of the 7 controls (6 hairpins designated N2, N3, N23, N4, N6 and N7 and 1 non-hairpin sequence designated N0) were expressed. N116 is a negative control sequence partially overlapping GAM116.

In the picture, test lanes including template are designated ‘+’ and the control lane is designated ‘−’. It is appreciated that for each of the tested hairpins, a clear PCR band appears in the test (‘+’) lane, but not in the control (‘−’) lane.

FIGS. 14A through 14C, when taken together validate the efficacy of the bioinformatic gene detection engine in: (a) detecting known MIR genes; (b) detecting novel GAM genes which are found adjacent to these MIR genes, and which despite exhaustive prior biological efforts and bioinformatic detection efforts, went undetected; (c) discerning between GAM (or MIR) PRECURSOR hairpins, and non-GAM hairpins.

It is appreciated that the ability to discern GAM-hairpins from non-GAM-hairpins is very significant in detecting GAM genes, since hairpins in general are highly abundant in the genome. Other MIR prediction programs have not been able to address this challenge successfully.

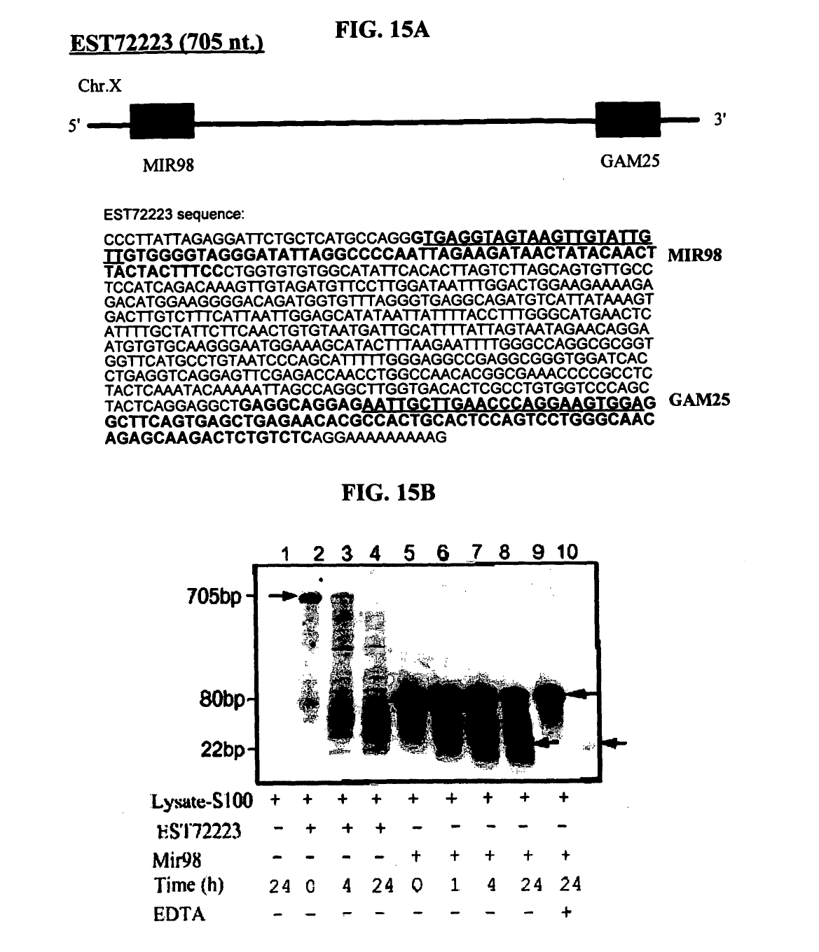

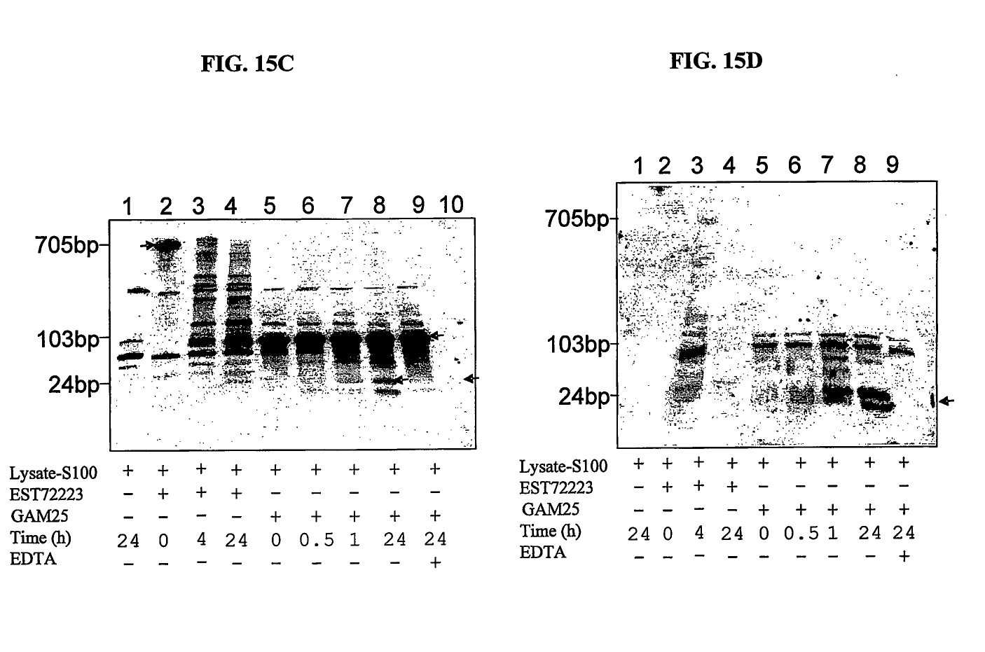

Reference is now made to FIG. 15A which is an annotated sequence of human EST comprising a novel gene detected by the gene detection system of the present invention. FIG. 15A shows the nucleotide sequence of a known human non-protein coding EST (Expressed Sequence Tag), identified as EST72223. The EST72223 clone obtained from TIGR database (Kirkness and Kerlavage, 1997) was sequenced to yield the above 705 bp transcript with a polyadenyl tail. It is appreciated that the sequence of this EST comprises sequences of one known miRNA gene, identified as MIR98, and of one novel human GAM gene, referred to here as GAM25, detected by the bioinformatic gene detection system of the present invention and described hereinabove with reference to FIG. 2.

The sequences of the precursors of the known MIR98 and of the predicted GAM25 are in bold, the sequences of the established miRNA 98 and of the predicted miRNA GAM25 are underlined.

Reference is now made to FIGS. 15B, 15C and 15D that are pictures of laboratory results, which when taken together demonstrate laboratory confirmation of expression of the bioinformatically detected novel gene of FIG. 15A.

In two parallel experiments, an enzymatically synthesized capped, EST72223 RNA transcript, was incubated with Hela S100 lysate for 0 minutes, 4 hours and 24 hours. RNA was subsequently harvested, run on a denaturing polyacrylamide gel, and reacted with a 102 nt and a 145 nt antisense MIR98 and GAM25 precursor transcript probes respectively. The Northern blot results of these experiments demonstrated processing of EST72223 RNA by Hela lysate (lanes 2-4, in 15B and 15C), into ˜80 bp and ˜22 bp segments, which reacted with the MIR98 precursor probe (15B), and into ˜100 bp and ˜24 bp segments, which reacted with the GAM25 precursor probe (15C). These results demonstrate the processing of EST72223 by Hela lysate into MIR98 precursor and GAM25 precursor. It is also appreciated from FIG. 15C (lane 1) that Hela lysate itself reacted with the GAM25 precursor probe, in a number of bands, including a ˜100 bp band, indicating that GAM25-precursor is endogenously expressed in Hela cells. The presence of additional bands, higher than 100 bp in lanes 5-9 probably corresponds to the presence of nucleotide sequences in Hela lysate, which contain the GAM25 sequence.

In addition, in order to demonstrate the kinetics and specificity of the processing of MIR98 and GAM25 miRNA precursors into their respective miRNA's, transcripts of MIR98 and of the bioinformatically predicted GAM25, were similarly incubated with Hela S100 lysate, for 0 minutes, 30 minutes, 1 hour and 24 hours, and for 24 hours with the addition of EDTA, added to inhibit Dicer activity, following which RNA was harvested, run on a polyacrylamide gel and reacted with MIR98 and GAM25 precursor probes. Capped transcripts were prepared for in-vitro RNA cleavage assays with T7 RNA polymerase including a m⁷G(5′)ppp(5′)G-capping reaction using the mMessage mMachine kit (Ambion). Purified PCR products were used as template for the reaction. These were amplified for each assay with specific primers containing a T7 promoter at the 5′ end and a T3 RNA polymerase promoter at the 3′end. Capped RNA transcripts were incubated at 30° C. in supplemented, dialysis concentrated, Hela S100 cytoplasmic extract (4C Biotech, Seneffe, Belgium). The Hela S100 was supplemented by dialysis to a final concentration of 20 mM Hepes, 100 mM KCl, 2.5 mM MgCl₂, 0.5 mM DTT, 20% glycerol and protease inhibitor cocktail tablets (Complete mini Roche Molecular Biochemicals). After addition of all components, final concentrations were 100 mM capped target RNA, 2 mM ATP, 0.2 mM GTP, 500 U/ml RNasin, 25 μg/ml creatine kinase, 25 mM creatine phosphate, 2.5 mM DTT and 50% S100 extract. Proteinase K, used to enhance Dicer activity (Zhang H, Kolb F A, Brondani V, Billy E, Filipowicz W. Human Dicer preferentially cleaves dsRNAs at their termini without a requirement for ATP. EMBO J. 2002 Nov. 1; 21(21):5875-85) was dissolved in 50 mM Tris-HCl pH 8, ≡l mM CaCl₂, and 50% glycerol, was added to a final concentration of 0.6 mg/ml. Cleavage reactions were stopped by the addition of 8 volumes of proteinase K buffer (200 Mm Tris-Hcl, pH 7.5, 25 mM EDTA, 300 mM NaCl, and 2% SDS) and incubated at 65° C. for 15 min at different time points (0, 0.5, 1, 4, 24 h) and subjected to phenol/chloroform extraction. Pellets were dissolved in water and kept frozen. Samples were analyzed on a segmented half 6%, half 13% polyacrylamide 1XTBE-7M Urea gel.

The Northern blot results of these experiments demonstrated an accumulation of a ˜22 bp segment which reacted with the MIR98 precursor probe, and of a ˜24 bp segment which reacted with the GAM25 precursor probe, over time (lanes 5-8). Absence of these segments when incubated with EDTA (lane 9), which is known to inhibit Dicer enzyme (Zhang et al., 2002), supports the notion that the processing of MIR98 and GAM25 miRNA's from their precursors is mediated by Dicer enzyme, found in Hela lysate. The molecular sizes of EST72223, MIR-98 and GAM25 and their corresponding precursors are indicated by arrows.

FIG. 15D present Northern blot results of same above experiments with GAM25 probe (24 nt). The results clearly demonstrated the accumulation of mature GAM25 gene after 24 h.

To validate the identity of the band shown by the lower arrow in FIGS. 15C and 15D, a RNA band parallel to a marker of 24 base was excised from the gel and cloned as in Elbashir et al (2001) and sequenced. 90 clones corresponded to the sequence of mature GAM25 gene, three corresponded to GAM25* (the opposite arm of the hairpin with a 1-3 nucleotide 3′ overhang) and two to the hairpin-loop.

GAM25 was also validated endogenously by sequencing from both sides from HeLa YM100 total-RNA “ligation” libraries, utilizing hemispecific primers as detailed in FIG. 13.

Taken together, these results validate the presence and processing of a novel MIR gene product, GAM25, which was predicted bioinformatically. The processing of this novel gene product, by Hela lysate from EST72223, through its precursor, to its final form was similar to that observed for known gene, MIR98.

Transcript products were 705 nt (EST72223), 102 nt (98 precursor), 125 nt (GAM25 precursor) long. EST72223 was PCR amplified with T7-EST 72223 forward primer:

5′-TAATACGACTCACTATAGGCCCTTATTAGAGGATTCTGCT-3′

and T3-EST72223 reverse primer:

5′-AATTAACCCTCACTAAAGGTTTTTTTTTCCTGAGACAGAGT-3′.

MIR98 was PCR amplified using EST72223 as a template with T7MIR98 forward primer:

5-′TAATACGACTCACTATAGGGTGAGGTAGTAAGTTGTATTGTT-3′

and T3MIR98 reverse primer:

5′-AATTAACCCTCACTAAAGGGAAAGTAGTAAGTTGTATAGTT-3′.

GAM25 was PCR amp using EST72223 as a template with GAM25 forward primer: 5′-GAGGCAGGAGAATTGCTTGA-3′ and T3-EST72223 reverse primer:

5′-AATTAACCCTCACTAAAGGCCTGAGACAGACTCTTGCTC-3′.

It is appreciated that the data presented in FIGS. 15A, 15B, 15C and 15D when taken together validate the function of the bioinformatic gene detection engine 100 of FIG. 2. FIG. 15A shows a novel GAM gene bioinformatically detected by the bioinformatic gene detection engine 100, and FIGS. 15C and 15D show laboratory confirmation of the expression of this novel gene. This is in accord with the engine training and validation methodology described hereinabove with reference to FIG. 3.

It is appreciated by persons skilled in the art that the present invention is not limited by what has been particularly shown and described hereinabove. Rather the scope of the present invention includes both combinations and subcombinations of the various features described hereinabove as well as variations and modifications which would occur to persons skilled in the art upon reading the specifications and which are not in the prior art.

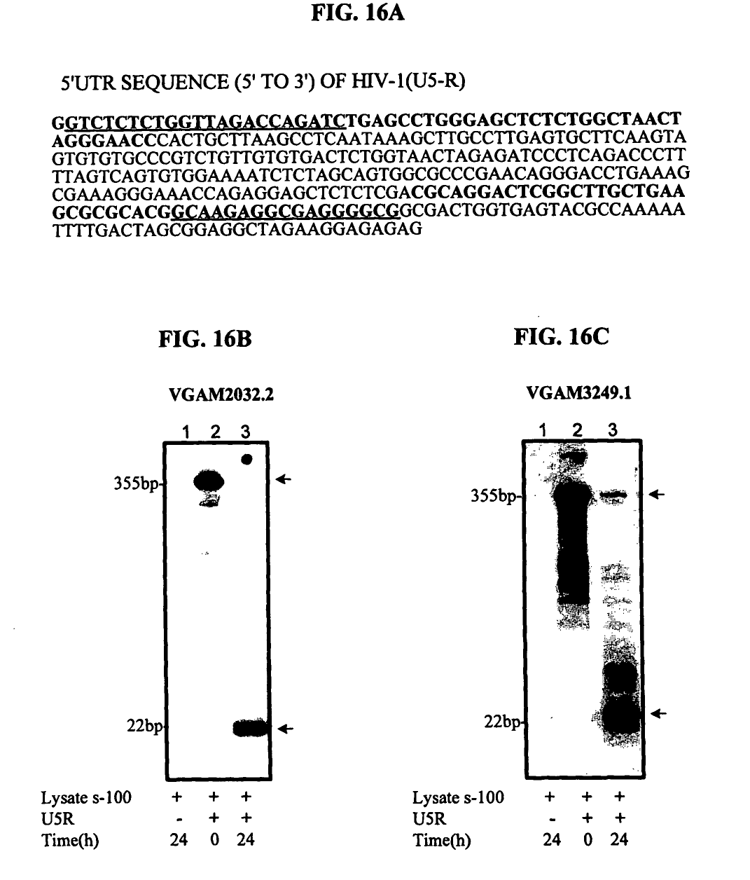

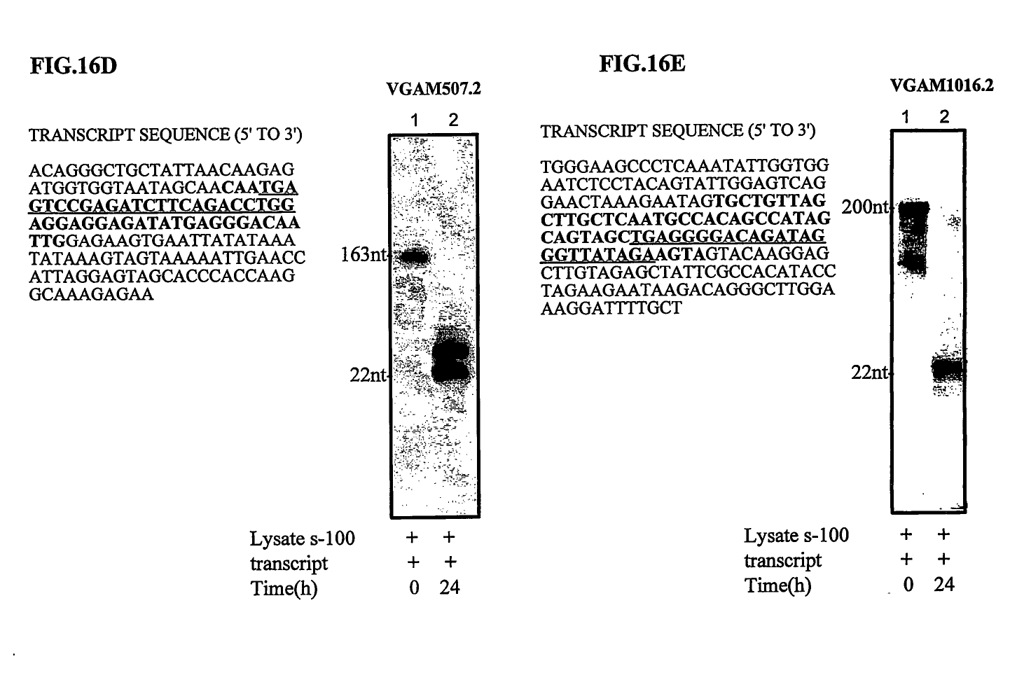

Reference is now made to FIG. 16, which presents pictures of laboratory results, which demonstrate laboratory confirmation of excision (“dicing”) of the bioinformatically detected novel VGAM HIV1 genes, herein designated VGAM2032.2, VGAM3249.1, VGAM 507.2 and VGAM1016.2 from their predicted precursors by their incubation in HeLa S-100 lysate as described in FIG. 15.

FIG. 16A presents the entire 5′-UTR of HIV1 (U5R) containing two predicted VGAM precursor genes, in bold; VGAM 2032 and VGAM3249. The bioinformatically predicted mature VGAMs are depicted with underscore, the 5′-most is VGAM 2032.2 and the second is VGAM 3249.1. VGAM 2032.2 matches the known HIV1 RNA structure named TAR to which the TAT protein binds (Nature 1987. 330:489-93).

FIG. 16B and FIG. 16C depict Northern blot analysis of VGAMs in U5R, hybridized with predicted mature VGAM oligonucleotide probes VGAM 2032.2, and VGAM 3249.1, respectivley. The molecular size of the entire U5R transcript, 355 nt, is indicated by arrow. The predicted molecular sizes of VGAM 2032.2, and VGAM 3249.1 are 22 nt and 17 nt respectively. The 22 nt molecular marker is indicated by arrow. Lanes: 1-Hela lysate; 2-U5R transcript in HeLa Lysate without incubation. 3-U5R transcript incubated overnight with Hela lysate.

FIGS. 16D and 16E present partial transcripts of HIV1 RNA reacted with predicted mature HIV1-VGAM oligonucleotide probes. In each figure, the experimental transcript sequence is shown, annotated in bold is the predicted VGAM precursor, and in underscore the predicted mature VGAM. Northern blot analysis of VGAM precursors for VGAM507.2 (FIG. 16D), and VGAM1016.2 (FIG. 16E). The transcript sizes are 163 nt for VGAM507.2 transcript and 200 nt for VGAM1016.2 transcript. The predicted molecular sizes of VGAM507.2 and VGAM1016.2 are both 24 nt. The 22 nt molecular marker is indicated by arrow. Lanes: 1—Transcript in HeLa Lysate without incubation. 2—Transcript incubated overnight with HeLa lysate.

It is appreciated that the sequence of the reacting bands in the foreseen sizes comprise sequences of novel VGAM genes, referred to here as VGAM 2032.2, VGAM 3249.1, VGAM 507.2, VGAM 1016.2, detected by the bioinformatic gene detection engine 100 of the present invention, described hereinabove with reference to FIG. 2.

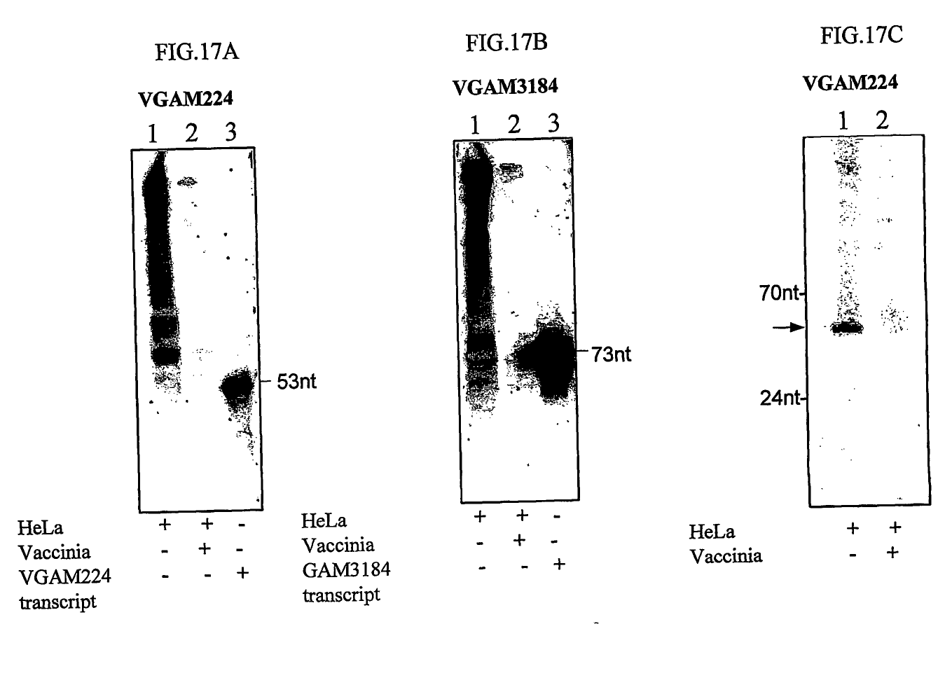

Reference is now made to FIG. 17 which presents pictures of laboratory results, which demonstrate laboratory confirmation of expression of the bioinformatically detected novel Vaccinia VGAM genes VGAM224 (FIGS. 17A and 17C) and VGAM3184 (FIG. 17B). HeLa cells were infected with 50 PFU Vaccinia Virus and total RNA was harvested after 3 days. Northern blot analysis of VGAM precursors in total RNA extracted from HeLa cells infected with Vaccinia Virus, lane 1, or HeLa uninfected cells, lane 2, and hybridized with predicted precursor DIG-labled RNA probes of 53 nt for VGAM224 (FIG. 17A), or of 73 nt for VGAM3184 (FIG. 17B) or with a 22 nt γ³²P-ATP-labled DNA oligo probe for predicted mature VGAM224.2 (FIG. 17C). A transcript of predicted sequence and size was run alongside as a size marker and as a hybridization control, lane 3 (except FIG. 17C). Arrow in FIG. 17C marks band of expected precursor size, 53 nt, reacting with mature 22 nt VGAM224.2 probe.

It is appreciated that the sequence of the reacting bands appear only in infected cells in-vivo and comprise sequences of novel VGAM gene precursors, referred to here as VGAM224 and VGAM3184, detected by the bioinformatic gene detection engine 100 of the present invention, as described hereinabove with reference to FIG. 2.

### DETAILED DESCRIPTION OF LARGE TABLES

Table 1 comprises data relating to the source and location of novel VGAM genes of the present invention, and contains the following fields:
GENE NAME Rosetta Genomics Ltd. gene nomenclature (see below)
VGAM SEQ-ID VGAM Seq-ID, as in the Sequence Listing
PRECUR SEQ-ID VGAM precursor Seq-ID, as in the Sequence Listing
ORGANISM Virus name
GENOME TYPE Genome type of the virus; dsRNA, dsDNA, ssRNA negative-strand, ssRNA positive-strand, Deltavirus or Retroid, as taken from ORGANISM definition by GenBank, NCBI.
GENOME STRUCTURE genome organization: circular or linear.
SOURCE_REF-ID Accession number of virus source sequence
SOURCE_OFFSET Offset of VGAM precursor sequence on source sequence
STRAND (+) positive strand, (−) negative strand
SRC Source-type of VGAM precursor sequence (see below)
VGAM ACC VGAM Prediction Accuracy Group (see below);

Table 2 comprises data relating to VGAM precursors of novel VGAM genes of the present invention, and contains the following fields:
GENE NAME Rosetta Genomics Ltd. gene nomenclature (see below)
PRECUR SEQ-ID VGAM precursor Seq-ID, as in the Sequence Listing
PRECURSOR SEQUENCE VGAM precursor nucleotide sequence (5′ to 3′)
FOLDED-PRECURSOR Schematic representation of the VGAM folded precursor, beginning 5′ end (beginning of upper row) to 3′ end (beginning of lower row), where the hairpin loop is positioned at the right part of the draw.
SRC Source-type of VGAM precursor sequence (see below)
VGAM ACC VGAM Prediction Accuracy Group (see below);

Table 3 comprises data relating to VGAM genes of the present invention, and contains the following fields:
GENE NAME Rosetta Genomics Ltd. gene nomenclature (see below)
VGAM SEQ-ID; VGAM Seq-ID, as in the Sequence Listing
GENE_SEQUENCE Sequence (5′ to 3′) of the mature, ‘diced’ VGAM gene
PRECUR SEQ-ID VGAM precursor Seq-ID, as in the Sequence Listing
SOURCE_REF-ID Accession number of the source sequence
SRC Source-type of VGAM precursor sequence (see below)
VGAM ACC VGAM Prediction Accuracy Group (see below);

Table 4 comprises data relating to host target-genes and binding sites of VGAM genes of the present invention, and contains the following fields:
GENE NAME Rosetta Genomics Ltd. gene nomenclature (see below)
VGAM SEQ-ID; VGAM Seq-ID, as in the Sequence Listing
TARGET VGAM target protein name
#BS Number of unique binding sites of VGAM onto Target
TARGET SEQ-ID Target binding site Seq-ID, as in the Sequence Listing
TARGET REF-ID Target accession number (GenBank)
UTR Untranslated region of binding site/s (3′ or 5′)
UTR OFFSET Offset of VGAM binding site relative to UTR
TAR-BS-SEQ Nucleotide sequence (5′ to 3′) of the host target binding site
BINDING-SITE-DRAW Schematic representation of the binding site, upper row present 5′ to 3′ sequence of the VGAM, lower row present 3′ to 5′ sequence of the target.
SRC Source-type of VGAM precursor sequence (see below)
VGAM ACC VGAM Prediction Accuracy Group (see below);
BS ACC Binding-Site Accuracy Group (see below)
TAR ACC Target Accuracy Group (see below);

Table 5 comprises data relating to functions and utilities of novel VGAM genes of the present invention, and contains the following fields:
GENE NAME Rosetta Genomics Ltd. gene nomenclature (see below)
TARGET VGAM target protein name
GENE_SEQUENCE Sequence (5′ to 3′) of the mature, ‘diced’ VGAM gene
GENE-FUNCTION Description of the VGAM functions and utilities
SRC Source-type of VGAM precursor sequence (see below)
VGAM ACC VGAM Prediction Accuracy Group (see below)
TAR ACC Target Accuracy Group (see below)
TAR DIS Target Disease Relation Group (see below)

Table 6 comprises a bibliography of references supporting the functions and utilities of novel VGAM genes of the present invention, and contains the following fields:
GENE NAME Rosetta Genomics Ltd. gene nomenclature (see below)
TARGET VGAM target protein name
REFERENCES list of references relating to the host target gene,
SRC Source-type of VGAM precursor sequence (see below)
VGAM ACC VGAM Prediction Accuracy Group (see below)
TAR ACC Target Accuracy Group (see below); and

Table 7 comprises data relating to novel VGR genes of the present invention, and contains the following fields:
GENE NAME Rosetta Genomics Ltd. VGR gene nomenclature
SOURCE START OFFSET Start-offset of VGR gene relative to source sequence
SOURCE END OFFSET End-offset of VGR gene relative to source sequence
SOURCE_REF-ID Accession number of the source sequence
STRAND (+) positive strand, (−) negative strand
VGAMS_ID'S_IN_VGR List of the VGAM genes in the VGR cluster
SRC Source-type of VGAM precursor sequence (see below)
VGR ACC VGR Prediction Accuracy Group (see below).
   The following conventions and abbreviations are used in the tables:

GENE NAME is a RosettaGenomics Ltd. gene nomenclature. All VGAMs are designated by VGAMx.1 or VGAMx.2 where x is the unique SEQ-ID. If the VGAM precursor has a single prediction for VGAM, it is designated by VGAMx.1. Otherwise, the higher accuracy VGAM prediction is designated by VGAMx.1 and the second is designated by VGAMx.2.

SRC is a field indicating the type of source in which novel genes were detected, as one of the following options: (100) DNA sequence, (101) RNA sequence. Sequences are based on NCBI Build33 of the viral genome annotation.

VGAM ACC (VGAM Prediction Accuracy Group) of gene prediction system: A—very high accuracy, B—high accuracy, C—moderate accuracy, D—low accuracy, as described hereinbelow with reference to FIG. 12.

BS ACC (Binding-Site Accuracy Group) indicates accuracy of target binding site prediction, A—very high accuracy, B—high accuracy, C—moderate accuracy, as described hereinbelow with reference to FIG. 14B.

TAR ACC (Target Accuracy Group) indicates accuracy of total GAM-target binding prediction, considering the number of binding sites a GAM has on the target's UTR; A—very high accuracy, B—high accuracy, C—moderate accuracy, as described hereinbelow with reference to FIG. 14B.

TAR DIS (Target Disease Relation Group) ‘A’ indicates if the target gene is known to have a specific causative relation to a specific known disease, based on the OMIM database. It is appreciated that this is a partial classification emphasizing genes which are associated with ‘single gene’ diseases etc. All genes of the present invention ARE associated with various diseases, although not all are in ‘A’ status.

VGR ACC (GR Prediction Accuracy Group) indicates the maximum gene prediction accuracy among VGAM genes of the cluster, A—very high accuracy, B—high accuracy, C—moderate accuracy, as described hereinbelow with reference to FIG. 14B.

1. An isolated viral DNA encoding:
RNA comprising about 50 to about 120 nucleotides,
wherein about 18 to about 24 nucleotides at the 5′ of the RNA are a partial inversed-reversed sequence of a sequence at the 3′ of the RNA, and wherein
a portion of the RNA is a partial inversed-reversed sequence of a portion of a binding site associated with at least one host target gene.

2. (canceled)

3. The DNA of claim 1 wherein the RNA is capable of modulating expression of the target gene.

4. (canceled)

5. (canceled)

6. The DNA of claim 1 wherein the untranslated region of an mRNA encoded by the target gene comprises the binding site.

7. (canceled)

8. A vector capable of expressing the DNA of claim 1.

9. A method of inhibiting translation of a host target gene comprising introducing the vector of claim 8 into the host.

10. (canceled)

11. (canceled)

12. A probe comprising a sequence complementary to a portion of the RNA encoded by the DNA of claim 1.

13. A method of detecting expression of a viral miRNA, comprising detecting hybridization by a probe of claim 12, wherein the probe comprises a sequence complementary to a portion of the miRNA.

14. (canceled)

15. (canceled)

16. (canceled)

17. (canceled)

18. A method for treating infection by a virus in a host comprising introducing to a host in need thereof a RNA comprising a sequence characterized by the following:
(a) the sequence is complementary to a portion of the miRNA expressed by the virus;
(b) the sequence is complementary to a portion of a binding site of a miRNA expressed by the virus;
(c) the sequence is complementary to a portion of a host miRNA characterized by increased expression with infection by the virus; or
(d) the sequence is complementary to a portion of a binding site in a host miRNA encoding a protein characterized by increased expression with infection by the virus.

19. (canceled)

20. (canceled)
